(19) United States
(12) Patent Application Publication
(10) Pub. No.: US 2008/0037018 A1
(43) Pub. Date: Feb. 14, 2008

(54) Methods and Systems for Determining and Measuring the Time of Death, Time, Condition and Liquid Content of and At Teeth or Materials

(76) Inventor: Andre Hoffmann, Dinslaken (DE)

Correspondence Address: WILLIAM COLLARD;COLLARD & ROE, P.C., 1077 NORTHERN BOULEVARD, ROSLYN, NY, 11576, US

(21) Appl. No.: 11/596,518

(22) PCT Filed: May 11, 2005
(86) PCT No.: PCT/EP05/04790

§ 371 Date: Nov. 15, 2006

(30) Foreign Application Priority Data
May 15, 2004 (EP) 10 2004 024 254.2

(51) Int. Cl. G01N 21/31; A61B 5/00

(52) U.S. Cl. 356/405; 356/402; 600/300

(57) ABSTRACT

Data are compiled by simulation before or during a process which a tooth or material can undergo or has undergone, and the data are then used for computing purposes and/or as reference data after an additional measurement data acquisition process in order to achieve one or more of the purposes of the disclosed methods. Data are acquired at the tooth or material by means of an instrument which can sense the spectral composition and/or the radiation path of electromagnetic radiation, in particular light, and/or by means of another conventional and known method for sensing a condition or liquid content.

If at all, all of the presently known forensic methods for determining regarding the time of death, allow for, at best, a rough estimation for the time of death. One of the most accurate and most frequently used methods in this field is the one, which is based on the cooling of the body temperature. Among other things, ambient temperatures, body girth and extent of the clothing have a serious influence on the rectally measured temperature of the deceased, which can be used to determine the time of death. In the forensic practice, the evaluation of rigor mortis, postmortem lividity, putrefaction and decomposition processes have little or no significance whatsoever for determining the time of death, and insect colonizations can only very vaguely and roughly, if at all, narrow down the time of death or the post-mortem period of the corpse.

For the purpose of the objective color description, the colorimetry is used in the industry with very different systems for the purpose of quality surveillance and in research in a material-scientific sense. Such equipment and systems (for example spectral photometers, tristimulus measuring devices, etc.) are designed for measuring at a planar surface and homogenous substances, such as plastics, automobile paints, print products, textiles. They generate a mostly standardized light, which is directed to the object or material, which is to be evaluated in terms of color. This object reflects the light in corresponding spectral composition, which it did not absorb, in which the light, for the purpose of a measurement, must hit the sensor of the measuring equipment, qualified to detection. Consequently, light hitting the sensor is processed in an exemplary manner, in that it hits photocells, is then converted into electric signals and finally converted into digital signals. Color measured values, values, values for establishing spectral curves, etc., for example, can be calculated from the digital signals. Usable data, which are connected downstream from the sensor, are resulted on each processing level. Experts refer to the arrangement of illumination and observer unit as measuring geometry.

Many of the currently available liquid or moisture content measuring methods are either quite inaccurate (e.g. electrical conductivity measurement), destroy or modify the material (e.g. thermo-gravimetry, chemical analyses, etc.) or the execution thereof has an enormous need for time (e.g. gravimetric, chemical liquid determinations etc.). Until now, the liquid or water content influence on the coloring of substances, in particular of non-liquid substances, has not been detected and evaluated, either scientifically or by means of a measuring process. The water or liquid-related influences on the color, in particular of non-liquid substances to a maximum extent of the color change were considered to be too low, to be somewhat difficult to differentiate, and to be too irregular and immeasurable. Presently known liquid or water content measuring methods thus operate, e.g., with color-amplifying influences (indicators) and in a substance-destructive manner (U.S. Pat. No. 6,043,096) or on the basis of the NIR spectroscopy (DE 198 30 720 C1), wherein the degrees of freedom, which are characteristic for each atom or molecule and, here, in particular, for the OH⁻ group, are used, and the molecules or atoms are stimulated to oscillations, which are characteristic for the OH⁻ group. To produce a result, even a quite inaccurate one, methods for the determination of time exclusively use a previously established combination of color and water content detection. At this point it is sensible to consult the applicant's yet unpublished studies with nine different measuring equipments and far more than 100,000 detected and evaluated values, on which, among other things, the methods and inventions are also based: It was possible to establish and quantify the relationship between liquid content, color, spectral composition of the light reflected by the teeth and its course of radiation (change), and the time factor within the liquid release and liquid absorption chronology. Teeth, but also materials or substances dry, release liquid or absorb liquid and change color in a characteristic manner. Color measuring values as well as the values for describing a spectral curve take up a trend and change with the direction for more than two to three weeks, in particular by simulation. In the experiments conducted by the inventor, it was possible, by simulation, to achieve an enormous unexpected or expectable precision increase in the measuring results or in the significance meaningfulness thereof. For example, the tooth, during the drying, gains brightness and the color measured values change their value in a directed way over longer periods of time. During air drying, the significance for the color measuring values and the liquid content in reference to the basic values of the liquid-saturated tooth is already present after an hour. Already at the onset of the air-drying phase, the brightness acts in a directed manner, while all other color measuring values assume the trends after 30 minutes. Under forced accelerated drying, comparable, yet accelerated trends can be accounted for. The processes (water and/or liquid absorption and release) are reversible. Depending on the type of tooth, a tooth weighs approximately between 0.510 g and 2,280 g and a front tooth weighs approximately 1,1277-1,1526 g and is in the studies determined in values of 1/1,000.000 g. The meaningful range lies below 1/10,000.00 g and a range of below 1/10,000.00% by weight of moisture content change can be detected and differentiated via color measuring values.

Due to this development and due to the chronological succession of the aforementioned values and of the curve progressions resulting therefrom, the inventor devised and described in the yet unpublished studies the “Pulpa-Dentin-Schmelzfluβ-Theorie” (“pulpa/dental nerve-dentin-dental enamel-flow-theory”), “Sog-Kapillarwirkungstheorie” (“undertow-capillary effect theory”), “Dentalklimaausgleichstheorie” (“dental-climate-balance-theory”), “Flüssigkeits-Farbeffekt-Theorie” (“liquid-color-effect-theory”), which state, among other things, that the drying does not only take place superficially at the dental enamel, but also reaches deep into the enamel, dentin and dental pulp and the tooth thus dries until it has reached a state of balance between its microclimate or the climate of its “natural spaces” (dental pulp, dentin tubuli, inter-crystalline regions, etc.) and its surrounding ambient climate, starting at the surface into its depth. In particular, the liquid flow occurs via capillary and undertow effect, very similarly to the biological example of the transpiration of a tree—evaporation of the liquid at its leaves—and its liquid transport via trunk and root system based thereon.

For each point of time of the drying process or liquid release process and/or liquid absorption process and/or water release process and/or water absorption process, there exists, for example, one or more of characteristic color parameters and/or a unique value combination and/or spectral curve and/or one or more of the values for describing a spectral curve, at which the point of time can clearly be identified (“dental clock”, “dental chronometer”, “dental time identification with tooth—color analysis”). In other words, one point of time of the drying period and/or of the process of water absorption and/or release and/or liquid absorption and/or liquid release and/or a condition changing process and/or a water content and/or a liquid content and/or a degree of moisture and/or (color) condition can be assigned to each measuring value or each measuring value combination and/or spectral curve and/or one or more of the values for describing a spectral curve. Furthermore, based on the observations within the aforementioned study, there exists a high dependency of the color measuring values and of the spectral curve, among other things, as well as of their development with the liquid release and absorption process on the already mentioned measuring geometry, on the measuring equipment which is used, on the standard observer angle, and on the standard light, among other things.

At this point, we must realize that manufacturers design the color measuring equipment for the measurements on planar surfaces, and not, at any rate, for such a complex tooth-curved surface. Depending on the measuring geometry, light transmitters and light sensors are in a spatial relationship and are coordinated by their design. Color measuring equipment thus has a construction-related coordination of light transmitter and sensor in the form of the equipment-specific measuring geometry. The measuring light of the instrument for making the measurement—reflected at curved surfaces—has irregular courses of radiation which are initially incompatible with the original technical concept of such color measuring equipment: A beam arriving at a tangential surface, which, due to the curvature of the crown is not located perpendicular to the axis of the device, forms a larger angle of incidence and allows for the angle of reflection to become the same. In this specific case of the measurement on naturally complexly curved teeth, the light emitted by the equipment arrives at several tangential surfaces of the curved tooth surface within the same measuring surface and is reflected by the measuring surface in very different directions. The light thus does not act upon the sensors with full intensity, but portions thereof are lost at non-detecting surfaces outside of the sensory regions. Inevitably, this leads to the loss of brightness and the color portions which seem to be brighter and more pleasant, in relationship to, e.g., the visually perceived color determination by means of tooth color samples used in dentistry, for example. In other words, the measuring results with color measuring equipment at teeth have hardly anything or nothing in common with the colors, which people visually observe, see and identify, as compared to the measurements and observations at a planar homogenous surface.

The tooth is a highly inhomogeneous highly structured grown structure. Selective absorption and remission of frequency portions of the light spectrum in the depth of the dentin core, as well as interferences, diffuse reflection and light dispersion at layer portions of the enamel close to the surface, light refraction, reflection, remission, and transmission in the region of all of the layers furthermore determine the measuring results. Regularly directed mirror reflections are created at these prismatic crystals. Factors, such as the layer thickness of the individual hard dental tissue, of the labial-oral crown diameter, the expansion of the pulp cavity also have an influence. The results, however, are strongly determined by the dentin, which shimmers through the relatively colorless and transparent enamel. The thickness of this enamel layer is responsible for the weakening of the intensity of the dentin color. A tooth thus becomes polychromatic. The relatively high light-permeability of the enamel is the result of the order in the form of a layered crystal design, which effects an expansion of the electromagnetic radiation, similar to an optical fiber. Tooth-typical shape and structural features form a reflection pattern. The composition of the dentin determines the hue and saturation. The extremely high individual structure of the natural tooth in the form of the tooth geometry, its crown and root curvature, and the uniqueness of the internal structure in the form of its layered design (enamel, dentin, dental pulp, relations and variations of the layer thicknesses), among other things, its individual crystal structure, individuality of the orientation, form and density of the nanometer-sized prism individually grown in the development phase, grid errors of the crystal design, the individual measure and the portion of organic and inorganic material, the composition and the chemical composition of those portions, etc. have a considerable influence on the courses of radiation and on the orientation of radiation of the reflected light beams and thus on the measuring results. The development by the successive exposure of these crystal prisms by means of liquid release as well as of the individual size of the dental pulp and quantity and of the composition of the crystal intermediate substance and thus the liquid content by the escaping liquid is thus not only highly individual but also differs in the light composition and radiation orientation and the results and result development are dependent on the equipment and measuring geometry, standard observation angle, standard light, etc. A change of the spectral light composition, but also a tooth-specific radiation processing and radiation processing tied to the individually grown and reflecting crystal prisms, among other things (e.g. refraction, transmission, remission, reflection, etc.) and directional change of the reflected radiation is thus connected with the liquid release as well as with the liquid absorption.

Only the radiation, which hits the sensors, can be generated into data. The light reflected by the tooth and/or passed, not absorbed by the tooth and/or the object, newly spectrally composed, running through, e.g., the most complex refraction, reflection, remission, transmission process, determines the measuring results and/or data.

The inventions and methods are based on one or more of the connections, which, among other things, have been proven by the aforementioned studies by the inventor and with which methods and/or portions thereof can be produced according to the claims: between the water content and/or liquid content and/or condition of the tooth and/or a material and the light reflected thereby and/or let through and/or electromagnetic radiation reflected thereby and/or let through and/or its and/or their spectral composition and/or courses of radiation of the light and/or of the electromagnetic radiation and/or of its color (objectively according to a measuring process and/or visually subjectively detected) and/or inventions and methods are based on the change of the water content (water absorption and/or release) and/or liquid content (liquid absorption and/or release) and/or condition change and/or change of the detectable data and/or changes of the light and/or changes of electromagnetic radiation reflected and/or let through by the tooth and/or by a material and/or electromagnetic radiation and/or its and/or their spectral composition and/or in the course of radiation and/or the change of the color detected by means of measuring processes and/or visually and/or of the condition in relation or in context to the process-related time factor and/or substance condition and/or object condition and/or material condition. The course of radiation refers to the direction of radiation and/or (e.g. spectral) portions thereof and/or to radiation components and/or to a pattern created therefrom. In the recording of reference data at a tooth and/or object or several teeth and/or objects and/or in the use at the tooth to be examined (e.g. tooth of the deceased, whose time of death is to be determined, or the reconstruction of the tooth color of a dried-up tooth of a patient, etc.) and/or “object”, a relation is to be established between the water content and/or the liquid content and/or the condition of the tooth and/or “object” and the data, gained from the light and/or other electromagnetic radiation reflected and/or let through by the tooth and/or object and/or its and/or their spectral composition and/or its and/or their course of radiation and/or the metric color measurement and/or visually subjective color selection (e.g. color samples, color pattern data) (objects and/or teeth with known liquid content and/or water content and/or the objects and/or teeth which represent a certain liquid content and/or water content are detected in one or more of the aforementioned data) and/or in the recording of reference data at a tooth and/or object or from several teeth and/or objects and/or in the use at the tooth and/or “object”, which is to be examined, a relation between water content and/or the liquid content and/or the condition of the tooth and/or of the “object” and the data, gained from the light and/or from other electromagnetic radiation reflected and/or let through by the tooth and/or object and/or its and/or their spectral composition and/or its and/or their course of radiation and/or the metric color measurement and/or visually subjective color selection (e.g. color sample, color pattern data) is established with the incorporation of the process-related time factor (e.g. liquid and/or water content absorption and/or liquid and/or water release) and/or in a further alternative according to the invention (claims), in the establishment of reference data at a tooth and/or object or several teeth and/or objects, and/or in the use of the tooth and/or “object”, which is to be examined, a relation and/or connection and/or context between the water content and/or the liquid content and/or the condition of the tooth and/or of the material and/or the data, gained from the reflected and/or passed light and/or other reflected and/or passed electromagnetic radiation and/or its and/or their spectral composition and/or its and/or their course of radiation and/or the metric color measurement and/or visually subjective color selection and/or a common condition detection method and/or water content detection method and/or liquid content detection method and the process-related time factor and/or the time period and/or point of time and/or time interval, or the like, within the process of the liquid absorption and/or liquid release and/or water absorption and/or water release and/or condition change and/or the change of the reflected and/or of the passed light and/or other electromagnetic radiation and/or of the its and/or their spectral composition and/or of the course of radiation and/or of the metric color measurement and/or of the visually subjective color selection is established. In one or more of the methods according to the claim, one or more of the established relationships and/or relations and/or context is used for the determination of the time of death and/or to determine a point of time and/or time period and/or to measure the liquid content and/or water content and/or the moisture and/or the condition by means of a data detection (e.g. based on electromagnetic radiation and/or, in particular, on the light) and/or to determine a water content and/or liquid content and/or condition, or the like, which prevailed at a definite other point of time and/or during a certain time period (e.g. prognosis, reconstruction, etc.) and/or to determine a point of time at which one or more predefined or determined and/or aspired data, or the like prevail and/or at which a predefined definite condition and/or water content and/or liquid content prevailed.

According to the claims, one or more of the recorded relationships or contexts or relations of the reference data can be used in that, by means of a metric detection or object with measuring processes and/or visually subjective detection by means of suitable instruments, and/or the liquid content and/or water content and/or a condition about, e.g., the light (e.g. spectral composition and/or course of radiation, etc.) and/or the color and/or color samples and/or electromagnetic radiation (e.g. spectral composition and/or radiation and/or intensities and/or pattern, etc.), or the like, are measured or detected and/or a condition and/or liquid content and/or water content and/or moisture is detected according to common or known methods (detection of the actual condition). According to the claims, not all of the relationships or contexts or relations and/or reference data must be recorded or detected. Depending on the use or later application, reference data for the spectral composition and/or for the course of radiation of the light and for the liquid content can be recorded and can be correlated, if the purpose is the measurement of the liquid content by means of “light” data. If, for example, a relationship of the “light” data to the time factor is to be used, for example, to determine the time of death, it is sufficient to connect reference data acquired from the light are connected with the time factor of the liquid absorption and/or liquid release process, etc.

According to the invention, the time of death can be determined by measuring the water content and/or the liquid content and/or the moisture by means of a commonly known method and/or measuring device by means of the amount of the residual liquid and/or a point of time and/or a time interval and/or the prevailing water content and/or liquid content and/or condition and/or reflected and/or passed light and/or other electromagnetic radiation and/or its and/or their spectral composition and/or its and/or their course of radiation and/or color (visually subjective and/or objectively metric), or the like, can be determined at a predefined and/or specific definite point of time and/or in a predefined and/or specific definite time period.

According to the invention, by means of detecting the passed and/or reflected light and/or electromagnetic radiation and/or its and/or their spectral composition and/or its and/or their course of radiation and/or color (visually subjective and/or objectively metric), a point of time and/or time interval and/or the prevailing water content and/or liquid content and/or the moisture and/or the condition and/or the reflected and/or passed light and/or other electromagnetic radiation and/or its spectral composition and/or its course of radiation and/or color (visually subjective and/or objectively metric), or the like, can be determined at a predefined point of time and/or in a predefined time period.

Alternative methods for recording reference data and/or for the detection at a tooth and/or object, which are to be correspondingly evaluated, can thus also exclusively use the established context between the process-related time factor and the data, gained from light and/or electromagnetic radiation and/or corresponding spectral composition and/or course of radiation and/or the liquid content, detected with one or more presently known or common methods for measuring the water content and/or liquid content and/or moisture measurement (e.g. thermogravimetric, gravimetric, radiometric moisture measurement, resistance moisture measurement, conductibility measuring principle, capacitive moisture measurement, CM-method/calcium-carbide-method, microwave moisture measurement, NIR-spectroscopy, thermography, use of the water activity, freezing point methods, compensation moisture measurement, Fischer titration, etc.) and/or by means of a corresponding instrument for detecting light and/or its spectral composition and/or its course of radiation and/or of electromagnetic radiation and/or its spectral composition and/or its course of radiation.

Further methods exclusively use the context between water content and/or liquid content and/or condition of the tooth and/or of the object and of the color and/or color measurement and/or color detection (visually) and/or the spectrally composed reflected and/or passed light and/or other electromagnetic radiation changed and detected in the course of radiation.

One or more of the instruments, which can be used, are thus, for example, at least one (light transmitter) light receiver system with at least one light receiver and/or, potentially, at least one light transmitter and/or at least one camera and/or at least one sensor and/or at least one detector and/or at least one image recording device and/or at least one image processing and/or at least one detection unit and/or at least one common liquid measuring equipment and/or water content measuring equipment and/or at least one of the previously known liquid measuring method and/or water content measuring method and/or color sample and/or comparative sample, etc., or the like. Detection examples are, among others, for example, CCD devices (charge coupled devices), ICCD devices (intensified charge coupled devices), EMCCD devices (electron multiplaying charge coupled devices), CMOS detector, camera, sensor, line camera, video camera, color camera, black-white or color (image) camera, camera, image processing, image recordation, NIR camera (near infrared) (wave range 900-1700 mm), IR camera (infrared), CCM coordinate measuring machine, CAD-CAM system, photodetector, in moving or non-moving images, UV-light camera, spectral photometer, color sensors, color sensor, color detectors, detectors, tristimulus measuring device, photocell, (fluorescence) spectroscope, micro-spectrometer, X-ray unit, CT, at least one imaging method, image-forming instruments (laminar microsensor arrangement), etc.

The condition of the method claim 1, refers to, e.g., the liquid content and/or the water content and/or the detected reflected and/or passed light and/or electromagnetic radiation and/or its and/or their spectral composition and/or its and/or their course of radiation and/or the color appearance, the color, the general appearance, etc., or the like.

All of the methods, i.e. those operating with reference data or standard tables, or the like, and/or which collect them or have collected them, as well as and/or those, which operate and/or exclusively operate at the “object”, which is to be examined, occur by simulation, which characterizes the methods. This simulation effects an enormous increase in measurement precision, which cannot be guessed beforehand, and an enormous increase in the ability to differentiate and/or discriminate the data and thus their significance and forms the inventive steps, among other things. Even individual color measuring values or one of them, for example, can now, even by themselves, describe the time of death. However, due to the theoretical mathematical monitoring of the probability, it is suggested to use more data and/or values, or the like, instead of less.

When the claims or the description use the term “simulation”, what is referred to is that a liquid content and/or water content and/or condition of a tooth and/or substance and/or a procedure and/or process and/or procedure and/or a portion thereof, through which a tooth and/or substance runs or can run (e.g. water and/or liquid absorption and/or water and/or liquid release, chemical and/or physical processes, etc.), or the like, is maintained and/or runs, if possible, under realistic and/or natural and/or lifelike and/or deliberate and/or given and/or desired and/or characteristic and/or adapted conditions and/or circumstances, or the like, or comes close thereto. One or more actions can thereby be implemented in order to change one or more of the influencing factors in such a manner so as to achieve or produce the aforementioned simulation.

The simulation is carried out for the recording of the reference data (e.g. for a standard table, a normal standard value table, a normal value curve, the device, the computer, the software, etc., or the like) at least one, but rather at as many samples as possible (e.g. teeth, objects, materials, items, etc.) and/or for the direct detection at the tooth and/or “object”, which is to be examined.

Simulation or conditions or circumstances with reference to the determination of the time of death pertains to the type of liquid, with which the tooth is and/or was brought into contact (e.g. saliva, nutritional liquids, nutritional components, means of the cause of death, seawater, waste water, etc.) and/or its consistency and/or its provenance and/or the liquid content and/or the water content and/or the condition of the initial state and/or the ambient milieu and/or the temperature of the ambient milieu and/or the moisture of the oral cavity and/or the humidity, and/or the body temperature and/or the type of the ambient milieu (gas, liquid, solids, e.g. concrete, sand, soil, loamy soil, etc.), changes to the teeth (“pink teeth”—phenomenon) and/or air pressure (e.g. altitude of the corpse) and/or storage of the tooth and/or use of the past medical history (anamnesis) and/or the patient's old medical findings, etc.

Simulation or conditions or circumstances with reference to substances pertains to the type and/or to the temperature of the ambient milieu and/or of the substance or of the involved substances and/or the humidity and/or the air pressure and/or the type of liquid and/or its consistency, and/or provenance and/or the liquid content and/or water content and/or condition of the initial state and/or of the ambient milieu and/or the concentration, and/or amount of one or more of the involved substances and/or reaction propensity and/or the storage of the substance, etc.

With reference to the conditions and/or circumstances in relationship to the liquid: The tooth should be stored in natural saliva of the same deceased, whose tooth it is and from whom the saliva can be acquired, e.g. at the location at which it collects and/or from one or more other deceased and/or living persons and/or in artificial or synthetic saliva and/or in a saliva-like substance. Artificial saliva can comprise the same components in comparable concentration, as the natural saliva or other components in other concentrations, which can also imitate the condition. Artificial saliva or artificial serum should, if possible, come close to the average density of the natural saliva of 1.01-1.02 g/ml, or should at least be in the tolerance range, which can be determined depending on the individual requirement, for example, and should, in addition to e.g. 95-99% water, also contain at least one of the following components: 33 or 10-130 mmol/l Na²⁺, 20-130 mmol/l K⁺, 50 mmol/l HCO₃, 34-80 mmol/l Cl⁻, Ca²⁺, or calcium chloride, dinatrium hydrogen phosphate, potassium chloride, sodium chloride, magnesium chloride, calcium chloride 0.05-1.22 g/1000 ml, magnesium pyrophosphate, potassium monohydrogen(—di)phosphate sorbic acid sodium benzoate, cholesterol mucins, pH 5.5-6.6 or 7.7 macromolecules, esterases peroxidases, glycoproteins, mucopolysaccharides, lysozymes, peroxidases, immunoglobulin A blood-type substances, e.g., α-amylase (ptyalin), proteins (mucins, enzymes, growth factors, immunoglobulins) and urea, hydroxyethylcellulose, carboxyethylcellulose, carboxymethylcellulose(—Na), polyethyleneoxide sorbitol, xylitol, linseed oil, ethyl alcohols, etc., and the like. Here, it should be required that the saliva comes as close as possible to the consistency of the saliva of the deceased, whose time of death is to be determined. Needless to say, the aforementioned composition or substance enumeration is exemplary, illustrative, or used as an example and that other compositions in substantial type and/or concentration or other substances certainly also lead to the goal and are thus also included in the scope of protection of this patent application or can also be carried out according to the claims of the methods, and shall thus also be included in the scope of protection of this application.

Medical findings should be carried out at the location where the corpse was found, whereby the position of the corpse and, in particular, of the head provides information as to where the saliva had or has accumulated, following the natural force of gravity. The saliva of the deceased can be collected here or directly at the excretory ducts of the salivary glands, in order to store the tooth therein.

The inclusion of the medical history and/or of the medical findings of the former attending physician and/or of the dentist, prior to the death, is advisable (alternative method), whereby the medical findings, such as, e.g., reduced or lacking or missing salivation (e.g. xerostomia, “dryness of the mouth”, sialopeny, oligosialia, asialia/aptyalism, etc.), the use of drugs (e.g. alpha and beta blockers, anti-depressants, anti-psychotics, anti-cholinergics, anti-histamines, sedatives, anti-Parkinson's drugs, etc.), syndromes (e.g. Sicca syndrome, Sjörgen syndrome, etc.), radiation or radiation lesion of the salivary glands, aplasia of the salivary gland, diabetes mellitus, hepatic cirrhosis, uremia, impeded nasal breathing, mouth breathing in connection with mouth dryness, the viscosity of the saliva (e.g. viscous, thin, or the like) are to be considered. For purposes of these new methods according to the claims, it should be required to establish the quantitative and qualitative saliva analysis by a general practitioner or by a dentist as a routine diagnosis in life, so as to be able to factor in these results into the diagnostic regarding the determination of the time of death.

The tooth, which is most suitable for this task, should be chosen for the determination of the time of death. For this purpose, a hierarchy can be set up for the teeth and teeth surfaces, which can be used for the determination of the time of death. In general, it can be said that the determination of the time of death can basically be carried out according to the claims at any of the available teeth and at any surface of the teeth. However, that tooth or those teeth, which are located further away from the foramen of the salivary ducts, should initially be used. It is proposed that, initially, one or more vestibular surfaces (front surfaces) of the upper jaw front teeth, followed by the platinal (side to the gum) surfaces of the upper jaw side teeth (premolar/molar), the platinal surfaces (gum surfaces) of the upper jaw front teeth (incisor/canine), the vestibular surfaces (sides facing the lip or cheek) of the side teeth (premolar/molar), and the lingual surfaces (facing the tongue) of the lower jaw teeth, are used. Occlusal surfaces (chewing surfaces), incisal edges (chewing edges) and, if present, proximal surfaces (surfaces to the nearby tooth) can also be used. Furthermore, those teeth, which, depending on the position of the head, are located at the deepest position, where, following the force of gravity, the saliva accumulates, and those teeth, which are farther away from this location, are to be preferred.

With reference to the conditions and/or circumstances in relationship to the ambient temperature:

During the first evaluation of the medical findings at the location where the corpse was found, the ambient temperature should be measured and recorded. The location where the corpse was found should also be examined as to whether and how far this temperature has been constant or could have changed over the post-mortem period of the corpse or the time of death. Ask these questions: is the deceased located outside or in a room? How much has the temperature outside or in the room at the location where the corpse was found decreased or increased (e.g. meteorology, night-time reduction of the heater, etc.)? To establish reference data and/or to determine the time of death at the tooth of the deceased, such temperature(s) and/or temperature fluctuations are to be included and/or considered.

With reference to the conditions and/or circumstances in relationship to the ambient milieu, and/or the humidity, and/or the air pressure of the air surrounding the corpse: the same as was explained for the aforementioned ambient temperature, holds true.

With reference to the conditions and/or circumstances in relationship to the temperature changes of the body:

In life, a human being has a body temperature of between 36.3-37.3° C. When death occurs (cerebral/brain death) and with the dying process, the body temperature drops considerably after six hours and decreases at 1-1.5° C. per hour, until the body temperature has assimilated to the ambient temperature. This change can be integrated in standard diagrams or the reference data for the determination of the time of death at teeth and/or materials, whereby these changes are established or acquired and utilized at corpses or at simulation objects.

With reference to the conditions and/or circumstances in relationship to the storage of the tooth:

The tooth can be kept in situ, i.e. in the mouth of the corpse and/or can be used with the head and/or skull and/or portions thereof and/or the entire corpse for the determination of the time of death. In this case, the liquid storage of the tooth must take place via a liquid container (solid or flexible, made of synthetic, plastic, silicon, etc., for example) which is adapted to the tooth or the mouth structure or the jaw by means of fastening elements (e.g. rubber membrane, hardening mass, etc.) and/or the corpse and/or the head and/or the skull with the tooth or the teeth is placed in liquid and/or liquid is added to the tooth, for example, in that it is moistened with liquid and/or coated with a liquid-carrying matrix.

The storage of the tooth takes place in vitro within a further alternative method according to the invention, i.e. outside of the body, whereby the body situation can be simulated, in that the root of the tooth, in correspondence with the natural conditions (largest part of the root is surrounded by bones and parodontium/dental ligamentous apparatus in an air unapproachable manner), is embedded in a retaining mass and/or separated from the ambient milieu by means of an isolating layer (e.g. on the basis of adhesive, alginate, polyether, silicone, preferably addition-curing silicones or polyethers, etc.,) and the tooth crown is left free at least at the location of the measurement, but, preferably, completely free and/or the mouth milieu and/or the mouth-head situation is simulated in that the head and/or the mouth and/or the back of the mouth and the pharynx (throat) and/or the face and/or portions thereof are realized, for example in the form of a dummy and/or the color design and/or geometrical design and/or structure-true design is to be as lifelike as possible or as similar to life as possible and/or the mouth milieu is simulated in that the dummy or portions thereof are correspondingly heated and/or the artificial mouth can be provided with liquid and/or with moisture of the mouth milieu. The structure surrounding the root of the tooth (e.g. bone structure, composition, liquid spaces, etc.) can also be simulated in a further alternative method, preferably by means of artificial bones or synthetic bones and/or natural bones (from the same corpse and/or from other dead and/or living creatures, such as, e.g. human beings or animals and/or masses, which can emulate the bone structure and the composition thereof (e.g. apatite, crystals, etc.), whereby, preferably the bone structure is to be similar to the natural bone (spongy and/or cortical) and/or the same is either naturally, e.g. jaw of a deceased and/or of this deceased and/or e.g. produced and held by means of a matrix (e.g. biological adhesive, glycoproteins, vessel, etc.). If the bone or the jawbone of the deceased, whose time of death is to be determined, is used, there are two possibilities: the tooth is either removed from the bone and placed in liquid (according to the method step from claim 1 and/or 2) or the tooth is left in the jaw and the tooth is brought into contact with liquid with the jaw and/or the head and/or the skull and/or the deceased and/or portions thereof and/or only the tooth, which is stuck in the jaw, is brought into contact with liquid.

An alternative method according to the patent for the determination of the time of death using teeth is thus based, e.g., among other things, on the phenomenon of the change of the spectral composition and/or of the internal and/or external structure of the tooth, tooth-geometry-related radiation angle change or change of the course of radiation of the light radiation and/or other electromagnetic radiation and/or change of the light and/or electromagnetic radiation reflected and/or let through by the tooth or by the teeth, which comes about if these teeth are subjected to a drying process. Teeth, which are exposed to atmospheric air, dry, become lighter and over long time periods quite characteristically and in a directed manner change the light and/or other electromagnetic radiation reflected and/or let through by the teeth, which hits the sensor. The light hitting the sensors is changed into data and/or values, which describe a trend. In the context of the liquid absorption of the tooth, the values and/or data perform diametrically to this (drying) process. When the patent claims or the description or the below correctly mention reflected or passed light, the color, and the color concept share in the results, and the spectral composition of the light hitting the sensors is also what is referred to, and when a tooth is mentioned, the same also holds true for the tooth portions or for several teeth, and vice versa. In this context, one cannot exclusively speak of color, but, correctly, of data acquired by the light hitting the sensors, which act directional and characteristically with the time of the liquid release and the liquid absorption, and highly individually for each tooth. The determination of the time of death can thus not only be described with one or with more of the many known color systems, but can also be described, for example, by means of all of the existing color systems or reference systems or by means of systems, which will be developed in the future, also of different types, but also by means of color system-independent values and data/data portions, etc.

According to the invention, the methods can be carried out with color measuring equipment (e.g. spectral photometer, tristimulus measuring device, etc.) with corresponding measuring geometry. However, the data determination is more accurate with the described light transmitter/light receiver system and/or transmitter/receiver system for corresponding electromagnetic radiation, possibly also by inclusion therein the course of radiation for the data acquisition. Instead, however, only one transmitter sensor arrangement (for electromagnetic radiation and/or, in particular, of light) with corresponding data processing, wherein the arrangement relation is maintained constant at least for the process of, e.g., the determination of the time of death at a tooth, which is to be evaluated, is required. Changes of the light refraction, remission, transmission, and reflection, etc., for example, with the liquid release, and/or absorption or the course of radiation can also be detected. If several light transmitters and/or several sensors are embedded in this definite relation, an information acquisition can be achieved via the processing, which is connected downstream, which was also achieved within an alternative according to the invention. If several sensors are used, the knowledge of the topographical location thereof and/or of their type allows for an information acquisition, in particular via the course of radiation and its change, which is connected with the liquid release or liquid absorption, which can be used for data acquisition, either by itself, and/or also with the spectral change is experienced. Only light hitting the sensor can be detected, e.g. in the intensity and/or in the spectral composition. It can also be envisioned, and as a further alternative for a solution according to the claims, that the radiation or its course of radiation, in addition to the topographical location also or exclusively takes place via sensors or image recordation, or the like, which are not effected by the main intensity of the radiation, but that they are sensors and/or detectors, which are qualified for this purpose, which follow or detect the course of radiation tendentious rather vertically to the course of radiation, or, for example, by means of detecting the intensity distribution, and/or the spatial resolution. Even with a use of limited light spectra, and/or of a limited light spectrum, and/or even of monochromatic light and/or laser light as a further alternative according to the invention, information can thus be acquired by the spectral composition, and/or by the courses of radiation, and/or by their change with liquid release or liquid absorption of a tooth or material. The same also holds true for corresponding alternatives according to the claims and for other electromagnetic radiation. It is also noted there that, according to the patent, each spectral section, and/or region of the light region can be used. The arrangement of the light transmitter—light receiver system and of the processing connected downstream can, according to the invention, occur within a device and/or an equipment, and/or a unit or several devices and/or equipment or units, etc., for example.

Usable data are created on each level of the processing, which is connected downstream from the sensor and/or the detector of the photocell and/or the camera and/or the “receiver” in a neutral manner.

When the claims or the description mention data and/or partial data and/or partial data (data parts), the same process would, in either case, be possible with only one data portion or with one date. However, due to the theoretical, mathematical view of the probability, it is advisable to use more data, instead of less data for the methods of the patent claims, if possible.

When the claims or the description mention data, and/or partial data and/or data portions (data parts) and/or reference data, this refers to, e.g., unprocessed or processed data, partial data, color measured number(s), color measured value(s), value(s) for describing a spectral curve, reference system-dependent and/or reference system-independent and/or color system-independent or color system-dependent data and/or date and/or one or more values of the liquid content and/or water content and/or moisture (e.g. % by weight, % by volume, g, mg/l, ml/g, rel.F %, % r.F., mg/g, ppm, ppb, etc.) and/or intensities and/or electrical and/or electronic-based values and/or one or more values for describing a spectral curve, and/or one or more values of the color measured values of one and/or more of the many hundreds of known color measured systems (e.g. CIELAB, CIELCH 1976, CIE94 of the Commission Internationale de l'Eclairage, Munsell, Hunter-Lab, DIN, RGB, NCS, etc., or the like) and/or of a system and/or pattern, comparative samples and/or data and/or terms thereof, which will be newly developed in the future.

The light or electromagnetic radiation reflected and/or let through by the tooth (portion) or by several teeth is to be detected and generated into data, in terms of establishing comparative data (reference data) with and specifically for the equipment and/or instrument and/or for the system, e.g. with corresponding geometry or detection arrangement, etc. and/or measuring conditions (e.g. standard observation angle, standard light, fluorescent light, light, etc., and the like), which is to be used.

For the previously mentioned reasons of the dependence of the results from the equipment, measuring geometry, standard observation angle, standard light, and manufacturer, etc., for example, a measurement of a tooth or of several teeth for the purpose of establishing reference data should, if possible, be carried out for the equipment or the system, which is used, individually with the equipment or the system.

A method example according to the patent for establishing reference data of a tooth and/or material and/or object and/or samples can be the detection of the data (liquid content and/or water content and/or data based on light and/or electromagnetic radiation and/or course of radiation and/or spectral composition and/or color detected visually and/or by means of a measuring process) and/or patterns (e.g. color patterns, comparative samples, or the like) along a drying or liquid release process or can also take place according to the invention via the liquid absorption of dry or drier teeth and/or materials. The time of detection within the chosen process can thus be assigned to the data and/or patterns. The development is described more accurately, the more data and/or patterns and the shorter the detection intervals.

The methods according to the patent are not limited, for example, by the time frame of the detection procedure, by chronological order, by the intervals and their chronological extents, and by the number of detections and of the teeth and/or materials and/or objects, etc., by the type and extent of the data and data detection and by and of the process, etc. The data (reference data) and/or value rows and/or curves etc., and the like, detected in such a manner can, in response to a current detection of the tooth and/or object, be assigned in data of a first evaluation. The data and/or patterns (e.g. comparative patterns, samples, or the like) or the value levels from current detections allow estimation regarding the time of death, e.g. in comparison via a standard curve and/or a computer program and/or via data processing, or the like, with the reference data and/or the sample (data), etc.

According to the patent, there is no limitation in which form the time factor and/or values and/or data, acquired from the light and/or electromagnetic radiation reflected and/or let through by the tooth and/or the teeth and/or object and/or spectral composition and/or course of radiation and/or common or conventional liquid measuring methods and/or water content measuring methods and/or visual pattern or samples determination, are put in connection. According to the patent, there is no limitation thus, this can be made, for example, via value tables, curves, comparative data, in the form of paper, directly in central or decentralized processing and/or allocation unit, in the measuring equipment, which, for example, immediately calculates the time factor and/or the water content and/or the liquid content, etc., or the like.

Detected data can be compared, for example, in alternatives according to the claims, even in consideration of influencing factors via a standard diagram, normal value tables, average value tables, curves, the measuring equipment, computer, data processing, etc., or the like, the time of death and/or time and/or liquid content and/or water content and/or condition etc., or the like, can be determined etc., or the like.

The acquirable data from a detection of the reflected and/or passed light and/or electromagnetic radiation and/or spectral composition and/or course of radiation by means of a suitable instrument and/or of the water and/or liquid content by means of a common instrument or an instrument (and/or method), which is known for this purpose, whereby the data previously acquired with this instrument are representative for a corresponding process-related time factor and/or water content and/or liquid content and/or condition, to which they are gaged or calibrated. In response to a renewed-detection or current detection at the tooth and/or object, which is to be evaluated, these data allow, e.g., the determination of the time of death and/or the of liquid content and/or of the water content and/or of the condition and/or determination or detection of a point of time and/or of time period in which a water content and/or liquid content and/or a condition prevails and/or predefined data can be measured and/or a water content and/or liquid content and/or a condition prevailing at another point of time, etc. By means of the data it collected, such an instrument is able to determine the time of death in a calculative manner, in a comparison or via a program and/or computer with the process-related reference data in an alternative according to the claims. It is thus possible that the forensic physician, for example, with such an instrument, collects data from one or more of the teeth of the deceased directly at the site of the crime or at the location where the corpse was found, or that he directly determines the time of death via the instrument or in combination with a computer. If influencing factors are included into the evaluation, this can occur, for example, via entries into this computer program. The instrument can, e.g., directly specify the time of death (e.g. display, printout, etc.). The computer program operates with reference data, which were recorded by simulation, e.g. under the most different conditions.

If the detections or the first detection of the data according to alternative methods are not carried out on-site, e.g. at the location where the corpse was found, but the teeth must be transported to the institute or special institute, and must sometimes cover long distances, the tooth or teeth should either be stored in a packaging, which is permeable to air and/or ventilated or has access to air (e.g. air pockets, microporous substance, or the like) or in such a packaging with an overpack, whereby there should be a space between the two, which space should, preferably, be controlled the temperature and/or air humidity, whereby the tooth further dries and the point of time of the first data detection in the institute is used as the basis for the calculation of the time of death. Or, in the alternative, the tooth is separated from the ambient milieu (e.g. air), for example by means of an isolating means (e.g. on the basis of alginate, silicone, polyether, adhesive, etc.) and/or the tooth is embedded in an almost humidity-impermeable mass (e.g. alginate, silicone, polyethylene, etc.) or is surrounded, e.g., with a material or a foil (e.g. plastic or synthetic, etc.) or an impregnation, whereby the time of the separation of the teeth from the ambient milieu by their packaging is defined and recorded as the point of time of the first postmortem measurement or the time of the separation forms the basis for the determination of the time of death, regardless as to whether or not this measurement took place at the time of packaging or first at the institute. If this detection first takes place at the institute, it is possible to consider a delay time, if the detection is not within a tolerable range.

Reference data can have been resulted at teeth of the living (living and/or dead teeth, extracted and/or in situ) and/or at the dying and/or the deceased.

The methods can also form the basis for a dental use. The drying in the dentistry generally takes place, relatively, with rolls of cotton or, absolutely, with dental rubber dam, a rubber membrane, which separates the teeth from the mouth milieu, and the tooth, which is to be operated, is kept try. For example a data pool established at teeth in vitro or in vivo, enables, after a drying has taken place, a reconstruction of points of time, but also or exclusively of prevailing teeth colors, i.e. also the reconstruction of the natural color of the liquid-carrying tooth prior to the onset of treatment. A use in the dental area would thus be the tooth color determination via a current detection, even after drying of the tooth with subsequent reconstruction of the tooth color and thus the answer to the question: What tooth color does the dried tooth have in liquid-carrying natural condition in the mouth of the patient or at any other point of time? By means of visual color determination, the dentist can also reconstruct the color condition, which the tooth naturally has in the mouth of the patient, from colors or, in a correspondingly metrical manner, from the data of the dried tooth or from the dried teeth (reference data), with the knowledge of the drying time. By means of one or more detectable data values (reference data) it could also be formulated up to which point no treatment-related drying-related pulp-damaging (nerve-damaging) effect is achieved and from what point it is achieved, so that the dentist, during treatment, is enabled to attend to the drying process in a controlled manner (monitoring), and to terminate the treatment, prior to damaging the pulp, or to add liquid to the tooth.

The determination of the time of death according to the method according to the claims by simulation allows for a maximally possible accuracy and precision and can be carried out in vivo or in vitro. Even the smallest color changes are made significant by the simulation. It is thus no longer absolutely necessary to measure the water content or the liquid content and, for example, the color as mutually conclusion-boosting factors. Instead, it is now possible to detect only the color or the water content or the liquid content or the spectral compositions and/or course of radiation and to come to highly accurate conclusions according to one or more of the methods and/or alternative methods according to the invention.

In the in vitro case and/or for the other alternative methods for detection at the object and/or for the increase of the accuracy, it is recommended to use a positioning system or a device, which prevents a result corruption, e.g. by means of measuring surface displacements and changes in the course of radiation by tilting or relocation of the equipment.

The device according to the claims enables an increase in precision, in particular of the methods according to the claims, and can be used in one or more thereof.

The (measuring) equipment, for example, is hereby maintained in relation via a fixed support or restraint system at the small measuring table at least in two dimensions (in particular for contact measurements) or in three dimensions (in particular for non-contact measurements). The measuring table is provided with an auxiliary device for applying a setting material or it contains the auxiliary device or forms a solid connection, for example to the auxiliary device, e.g. relief, frame, etc., for example by means of adhesion, screwed connection, etc. Equipment and support or restraint system can be interconnected with one another and/or removed from one another by means of screwed-in connection, plug-in connection, etc. The same holds true for the transitions from the small measuring table to the support or restraint system and/or from the small measuring table to the auxiliary device, which also transition into one another in combination, for example, or which are immovably mounted on one another by means of a screwed connection, plugging, etc. and which can be attachable and/or removable. This mounting can be realized, for example, via an attachment, also with a bolt, by means of a screwed connection, also with a groove, etc. Furthermore, according to the invention, even between these and within these components, there is a possibility for a separation and connection device, which, however, in response to a connection and re-connection, must ensure a fixed relation in the two or three correspondingly required dimensions. The support system, on its part, is mounted and/or can be mounted or forms a unit with the measuring system and/or with the equipment and/or with the light transmitter-sensor system etc., or the like. This auxiliary device comprises a relief and/or negative depressions, for example, or positively protruding portions, for example, etc. A frame is conceivable, which can accept the setting mass, would also be possible here. The auxiliary device is characterized in that a setting material (fixing mass) is introduced into it, is pressed onto it, assumes an non-disposable fixed relation to the measuring table, can be removed therefrom, but can also again be brought exactly to the same location in terms of a key-lock system. According to the claims, the setting material (fixing mass) must thus initially be liquid to viscous and is to become soft or elastic, but better yet solid to/or hard over time by means of reaction and/or setting. According to the patent, a liquid to viscous mass, into which the tooth (portion) and/or the material and/or the object is placed, and which mass, over time, thickens and/or becomes hard and/or soft-elastic to hard-elastic or hard is thus put on the auxiliary device. By positioning a tooth and/or object and/or several teeth and/or objects on or into the setting material, the object becomes the male part, the setting mass become the female part at this location.

The auxiliary device (relief, protrusion, frame, etc., or the like) and the tooth and/or the object, which is to be detected, as positives leave a negative impression or positive impression in the setting mass and thus form a female-part/male-part system according to a key-lock-principle, which enables corresponding positioning and/or repositioning.

For the case that the object, which is to be detected, must be supplied between two detection processes, e.g. for a further use, or the like, it would also be possible to remove not only the object alone from the setting mass, but the object with the setting mass block together from the auxiliary device, with which it is connected or can be connected via a key-lock principle, whereby an accurate repositioning of the object together with the setting mass block enables the relocation of the accurate equipment-object relation is made possible in an alternative according to the claims. Separations are also possible in all areas between measuring equipment and the object, which is to be examined, as long as the fixedly provided equipment-object relation or detection situation can be realized via, for example, attachments, male part-female part systems, a screwed connection in connection with a groove, etc.

The tooth, which was removed from the deceased and/or any material and/or object or a portion thereof, for example, can be placed into this setting mass. On one side, the setting mass with the auxiliary device (relief, frame, or the like, for example) and the object and/or the tooth and/or material also forms a positive-negative situation with the setting mass, which operates according to the key-lock principle. If the setting mass has cured and/or has become elastic to solid, there is a possibility to remove the object and/or the tooth from the setting mass, if necessary, and/or to also remove the setting mass from the auxiliary device and, if required, place it back to precisely the same position. This may become necessary, for example, for a below-mentioned liquid storage of the tooth and/or material. According to the patent, a completely hard setting mass can also be used. However, when the object is placed, the removal of the object from the setting mass is not to be hindered by regions located below the object, which occurs, if the object is to deeply embedded in the setting mass. Furthermore, according to the patent, there is also a possibility to store the object in the liquid together with a setting mass, which is impervious to liquids, or even to place further components of the positioning system into the liquid storage, for example the auxiliary device, the frame system, the small measuring table, the support system or portions thereof, etc. However, it is a requirement here that these components, wherever their separation is made possible from the remainder of the positioning system, allows for their accurate repositioning, e.g. by means of precision attachments, male parts-female parts systems, screwed connections with groove, etc., to the remaining device. The measuring equipment-object relation, measured in a non-contact mode, requires immovability in all three dimensions from the device. For measurements in contact mode, it requires the observation of this relation in only two dimensions. In this case, the equipment must be moved to the object in contact mode, for example via precision rails, attachments, plug-in systems according to the key-lock-system, etc. If the measuring equipment operates in contact mode, the support system makes it possible to allow the measuring equipment to move in a controlled or directed manner on the tooth or the object, for example via attachments, hinge, male parts-female parts systems, etc., or the like.

However, a determination of the time of death is also possible at the tooth in vivo at the corpse, which may still be carrying the tooth. As already stated, a specific process is required for this purpose, e.g. the use of a liquid-carrying system, e.g. a container, which can be adapted and sealed at the tooth or at the tissue surrounding the tooth. For the detection of the reflected and/or passed light and/or other electromagnetic radiation or of the special composition of the light and/or of other electromagnetic radiation and/or of the course of radiation, it would be necessary to require that the body, or at least the head, is fixed, for example as is the case with CT-recordings and/or, better yet, as with the tumor or radiation therapy, where the head is screwed in and/or fixed at the head bone and/or cranial bone via the tips of a fixing equipment. This fixing equipment must be fixed and/or fixable (repositionable) at the table, on which the corpse is placed, for example, and/or must form a unit with the table. In the alternative, the measuring surface can also be retrieved via the scanning or detection of the shape of one or more of the teeth and/or measuring surfaces and/or of the head and/or of the body (e.g. laser, cameras, also from different directions, for example, Streifenlichttopometrie (3-dimensional photorealistic and orthochromatic documentation of body surfaces), CT x-ray, etc.) and/or a portion thereof and thus the mapping of the surface and/or of the measuring surface of one or more of the equipments and/or the instruments according to the claims or of one of the detected portions thereof via a computer program and/or via a scanning system and/or detection system and/or repositioning possibility, e.g. robotic arm, rails, attachments, etc. and it can be brought back precisely into the same relation to the measuring surface or it can be reconstructed via the data, which are detected from another position, in that the topographies are placed on top of one another (bring the topographies in congruence) and the data are thus changed correspondingly. Topographies in connection with the image information and/or the measuring data of corresponding equipments or instruments according to the claims allow for a reconstruction of the data in relation to the position of the head and/or of the body and thus of the position of, e.g., the tooth, which is to be evaluated or of the measuring surface, in particular by using a neuronal network.

Depending on the characteristic and/or the individual requirements, the measuring surface and/or the measuring spot can also be freely chosen. If the measuring surface is retrieved exclusively via a fixation, the instrument and/or the equipment conducting the measurement are to be attachable (repositionable) and/or attached at the fixing equipment and/or to form a unit therewith. A fixing for the detection of data with, e.g., a sensor, detector, color measuring equipment, transmitter-receiver-system for electromagnetic radiation, light transmitter and light receiver system and/or a camera and/or an image recording and/or image processing system, etc. is not necessary. A fixing of object or tooth in relation to the instrument increases the accuracy of the results and measurements and the differentiability of the data and can be carried out depending on the requirement and desired accuracy. It is to be noted, however, that several recordings by means of a camera and/or image recording etc., for example, of different positions and/or in at least approximately predefined and several positions, would be better.

One or more measuring points or measuring surfaces can be used for the data detection. According to method claim 1 for the determination of the time of death, a first postmortem detection of the above-mentioned data and/or patterns and/or a portion thereof takes place after death has occurred and the registration of the when-data (e.g. the time, the day, the year, etc.) of this first data detection and/or sample detection takes place most mortem in the context of the forensic procedure. This first postmortem data detection and/or sample detection can be carried out as a one-time detection or as a repeated detection (e.g. data row, sample row, etc.) over a period of time. The latter possibility includes an additional information acquisition and is recommended.

These data and/or pattern/samples (data) can now be compared in an alternative method with the data from a reference data detection (e.g. comparative data pool, comparative pattern/samples pool, or the like), and, if present, can allow for an estimation of the time of death (according to method step from claim 2).

According to claim 1, the tooth is placed into a liquid (e.g. water, better yet a saliva-like substance, artificial saliva, synthetic saliva, or the like) or is brought into contact with a liquid until or closely to a liquid saturation (or into a tolerance range, chosen depending on the degree of accuracy, which is required), the condition in which it was present or must have been present in the mouth of the individual, while the individual was alive, and the condition at which the tooth has or had very characteristic data and/or patterns, whereby this simulated condition is also to be detected in data or pattern(s).

These data and/or the difference between these data and/or patterns and the data and/or patterns of the first postmortem detection, compared with the comparative data or reference data and/or the reference patterns, also allows for information regarding the time of death according to an alternative of claim 2.

This process of the liquid absorption can occupy 2-3 weeks and it is suggested that it should be carried out at least that long and/or, if possible, the data development and/or pattern development thereof should be monitored. However, the liquid storage can also take place over shorter time periods. The liquid-carrying tooth, while its carrier is alive, has a reflected light spectrum, which is quite characteristic, and generates characteristic courses of radiation and corresponding data or values. When death occurs, these change. The onset of this phenomenon also takes place with the removal of the tooth from the liquid and the process of the change, e.g. of the spectral light composition, the composition of electromagnetic radiation and/or of the course of radiation, etc., and the data change after death has occurred, which is connected therewith, can also be simulated or reconstructed by the drying of the tooth. Consequently, at least one detection, but better yet several detections of the data and/or of the visual pattern determinations is carried out according to method 1 during the drying process until the data and/or patterns and/or the data and/or patterns of the first detection at the tooth of the deceased have been reached or approached. If this traceable drying is an air drying and if it is carried out under comparable conditions (e.g. ambient temperature, humidity, etc.), such as the conditions, which acted on the tooth after death had occurred until the first detection, then the time of the data detections and/or pattern determination of the tooth in liquid-saturated condition to the reached or tolerable approximation on the data and/or patterns of the first detection after death occurred corresponds to the time when death occurred to the first postmortem detection and/or first data determination after death occurred. The elapsed reconstructable drying time or liquid release time, from the liquid-saturated condition of the tooth to the condition, at which the values or data and/or patterns approach and/or resemble and/or are in the predefined tolerance range of those data, which had been detected at the first detection after death occurred, corresponds to the time or the time period, which passed from the occurrence of death to the first detection after death occurred. Starting at the point of time of the first postmortem data detection and/or pattern detection, this elapsed time (according to the reconstructed drying time or liquid release time), calculated back into the past, results in the time of death or the point of time at which death occurred according to method 1.

If drying takes place under thermal influences, i.e. a heated tooth is dried, the determination of a result must result from the experience with these methods and/or via corresponding reference data collection and/or reference pattern collection (e.g. computer program, measuring device-internal calculation, standard table, standard curve/standard curve collection, etc., and the like) and/or take place via a chronological correction factor, which can be derived therefrom, whereby further alternative methods according to the claims are described. Correction factors or reference data and/or reference patterns based on simulation are also required for the detection under conditions, which differ from those conditions of the normal average postmortem situation of the tooth. One reference tooth or several reference teeth and/or reference objects, detected via very different temperatures in the data and/or via patterns, can thus supply reference data for different or very different postmortem situations, which are based on temperatures. Reference data, raised by simulation, for example, of the air moisture of the mouth milieu, and/or of the ambient air and/or of the temperature of the corpse and/or the change thereof and/or of the air pressure and/or of the liquid contents of the tooth and/or of an object and/or of the ambient structures and/or of the consistency and/or of the composition of the liquid or of the saliva and/or of the flow thereof, which runs dry with the occurrence of death, etc. also result in a reference data collection developed and/or usable according to the claims.

If the mouth of the deceased is open when death occurs or after death has occurred, the measurable relevant drying phase begins immediately. At room temperature, this drying period extends far beyond more than two weeks. The determination of the hour of death within this time period is considered to be possible and the determination of the time of death within the first days of drying could be by far more accurate.

By excluding the detour via the transmission and/or reflection light detection and/or via other electromagnetic radiation, or the like, it is also possible to formulate the time of death directly via the detection of the liquid content of the tooth or tooth portion, whereby the liquid content is detected by means of common or known water content and/or liquid content and/or moisture measuring devices and/or equipments or methods. The residual liquid content of the tooth (tooth portion) (residual liquid) alone allows for a rough estimation of the time of death. The more residual liquid, the less time has passed after death has occurred. With the help of reference data acquired at comparative teeth, the amount of residual liquid thus determines the time of death. In the run-up, data pools (weight, liquid content, % by weight, % by volume, g., ml/g, etc., for example) can be established at sample teeth and a statement regarding the time of death can be made by means of the first measurement after death has occurred and/or with the data of the liquid-saturated condition: According to claim 2, the time of death can thus be determined via the reference data previously established at other teeth with reference to the time of the drying and/or liquid absorption process (e.g. standard value collection, computer program, etc.).

Only a simulation or reconstruction of the postmortem time, according to claim 1, allows for a highly accurate time calculation. 1. First postmortem detection/detections after death has occurred, 2. Liquid storage of the tooth, 3. Detection/detections prior to and/or during the liquid release process to the data of the first measurement or to an approximation thereon. The elapsed reconstructed drying time or liquid release time, from the liquid-saturated condition of the tooth to the condition, at which the values or date come close to or are the same as those, which had been detected at the first detection after death had occurred, corresponds to the time or the time period, which lapsed from the occurrence of death to the first detection after death had occurred. Starting from the time of the first data detection, this lapsed time, calculated back into the past, results in the time of death.

A determination of the time of death by means of (tooth) color patterns, as they are used in dental offices for the color determination and matching of dental prosthesis on natural teeth, represents a further alternative according to the claims and is already described (e.g. via reference patterns and/or corresponding reference pattern data and/or via the reconstruction of the postmortem phase with corresponding determinations).

The subjectively detected and evaluated optical development of the brightness can be compared with the one according to a measuring process. There are considerable differences between the visual detection and the detection by means of a measuring process for the color development beyond the brightness. However, directional trends also exist here. The explanation lies in the layered arrangement of the tooth (enamel, dentin, pulpa). The brightness is assigned to the enamel, the uppermost layer of the tooth. The coloring, hue (e.g. blue, red, yellow, etc.) and saturation is assigned to the dentin. The component determining the brightness, the enamel layer, becomes lighter with an increasing drying and places itself, like a veil, across the dentin layers of the depth, which determine the actual color. The applicant calls this phenomenon the “Schichtungsphänomen” (“Layers-Phenomenon”). Visually, the bright colors of the dentin layers cannot be noticed very much, the brightness (enamel) comes to the fore, the characteristic color (dentin) moves into the background in an increasingly unsaturated manner. This is entirely different with the color detection, for example with color measuring equipments. A flash of light, which shoots out from the light transmitter of the instruments, almost completely penetrates the upper enamel layer and reaches, although increasingly weakened, to the dentin core and further into the depth. The reflected evaluation light, which hits the sensors of the measuring equipment, thus contains considerably more information from the internal of the tooth, than a human observer would be able to accomplish (“Layers-Phenomenon”).

However, a directional behavior in the color development can here also be recognized in response to the visual subjective pattern determination. Likewise, in a further alternative according to the claims, a pool of comparative patterns or of data describing the patterns, however, is established, to which the time factor of, e.g., points of time after death has occurred and/or within the water absorption and/or water release chronology and/or liquid absorption chronology and/or liquid release chronology and/or condition change etc., to which the patterns in the tooth in the corresponding condition (or the corresponding tooth) come closest with reference to the color, are assigned or can, consequently, be assigned. The attribution takes place via already present (color) patterns or via patterns, which are simulated to the (tooth color) conditions of corresponding points of time or time periods or which are produced for them via patterns. The simulation of the process after death has occurred in the form of a chronological drying process, with the use of color patterns for detecting the color of one or more teeth and its effect is realized as an alternative method for the determination of the time of death according to method 1 in a chronological process: first postmortem visual pattern determination(s) (1.), liquid storage of the tooth (2.), one or more pattern determination(s) and subjection of the tooth to a drying period with accompanying pattern determination(s) (3.), are determined in a highly accurate manner. In this case, the patterns must not already be connected with the time factor beforehand, but can also be assigned to this time factor in the current diagnostic for the time of death.

The patterns or pattern data, chosen or determined in a, e.g., the first postmortem visual determination and/or visual determinations at the liquid-saturated tooth, compared with time-attributed patterns (reference patterns, comparative patterns or comparative data pool) and/or tooth color spaces and/or pattern-specific and/or tooth color space pattern-specific data or times assigned thereto, allow for an estimation of the time of death according to an alternative of the method 2. This is likewise made possible by the difference between the patterns from the visual first determination, in terms of color, after the occurrence of death, and those visually determined at the liquid-saturated or liquid-carrying tooth. This difference, and all of the pattern determinations is/are also made possible in the form of data, times or information contents, describing the pattern or tooth color space pattern, also with reference to the chosen pattern. A comparison of the first visual pattern selection and/or the difference between the same and the pattern selection at the liquid-saturated tooth with the comparative pattern or with the data or time data connected with these selected patterns, also allows for a rough estimation of the time period of the time of death.

If, after a certain period of time of the liquid release process, whereby at least one, or better yet several patterns are visually selected and assigned to the determination time within this process, prior to and also during the liquid release, if the chosen pattern approaches the pattern or a pattern from the same tooth color space, chosen for the first visual detection after death has occurred in terms of color (tolerance range) or if the same pattern was chosen, the time required by the liquid release process to bring the liquid-saturated tooth (pattern or tooth color space pattern prior to liquid release process corresponds to the natural tooth color of the living being or comes close thereto) into this color condition (pattern after occurred liquid release or partial release corresponds to the tooth color of the deceased at the time of the first determination and at the time after corresponding extra-oral completed liquid release) in response to air drying according to the lapsed time starting at the time of death to the first determination after death has occurred. If this time is counted or calculated back from the point of time of the first detection into the past, the time of death is determined. If the liquid release is accelerated via an increased temperature, or reconstructed under other conditions, a correction factor or reference data collection, established by corresponding simulation, must be used.

According to the patent, there is no limitation, for example with reference to the material selection, type, form, and composition, appearance and extent of those patterns, which can be removed or fixedly connected with a palette, either in an isolated or connected manner. According to the patent there are, furthermore, no limitations in the chronological process, in the amount of the detections and the succession thereof, as well as in their chronological sequence and their chronological distances from one another, etc.

However, the (tooth) color patterns, for example, of the presently available color palettes are insufficient to completely describe the changes in terms of color. According to the claims, an expansion of those color palettes with additional color palette samples or color patterns or comparative patterns in all color region, for example higher and/or lower brightness, e.g. of the green region, red region, yellow region, blue region and further hues, etc. and in the region of greater and/or lesser saturation than previously realized, for example in dental color shade guides, is required. The requirement of the color samples would be sensibly described within a (tooth) color space. N. Hall (EP 0591 958 A1) describes a tooth color space, established and realized for natural teeth of a living being. An alternative according to the patent uses these foundations and supplements the tooth color space of living individuals, which, with reference to the tooth color space of living and dead individuals, is highly limited, with exactly these enormous tooth color regions of teeth, the individuals of which are deceased. Strictly speaking, each point of time after the occurrence of death or each point of time after dry storage or drying of the tooth or of an object has its own (tooth) color space, which, according to the patent, must be established in an equipment-specific, measuring geometry-specific, standard observation angle-specific, standard light-specific manner. A design of the (tooth) color patterns according to tooth color spaces, which are assigned to the points of time or time periods of the liquid release and/or liquid absorption process ((tooth) color spaces, which are assigned to condition and/or time (of death) and/or post-mortem period of the corpse) is also realized according to the claims. The color detection for establishing the samples and/or the (tooth) color spaces takes place metrically and/or visually. Each (tooth) color space, consisting of those teeth, which are most frequently found at that point of time, is to be assigned to a point of time of the liquid release and/or of the liquid absorption process and/or of condition changes or can be assigned thereto. As the time of the liquid release increases, this (tooth) color space expands and is relocated into brighter regions. (Tooth) color samples or corresponding arrangements or entire shade guides, even of a common type, are to be expanded in all directions of the color space, for example. According to the invention, this shade guides can be established visually subjectively as well as by a measuring process a comparison with natural teeth, also via auxiliary means, for example, such as photos, camera, image processing, color measuring equipment, etc. A corresponding point of time (time period) and/or liquid content is assigned or can be assigned to each color pattern or color pattern group of, e.g., the time-specific or condition-specific (tooth) color space of the (tooth) shade guides, which originates from the analysis at natural teeth or objects and/or analyses at teeth and/or objects passing through a drying process or water and/or liquid release process and/or water and/or liquid absorption process at defined points of time.

The expansion of the color samples should take place on the basis of naturally occurring coloring and on possible colors of natural teeth and/or objects according to different points of time of the liquid release and/or liquid absorption behavior and/or different liquid contents. The connection of the time factor and/or of the condition with the coloring within the same color sample also corresponds to the patent. A substance-specific “gaging” of the comparative patterns or comparative samples to substances with known, for example, water content, liquid content, condition, etc., for example, without detection within a process and/or the use of these visually and/or metrically “gaged” patterns or samples for one or more purposes according to the invention also corresponds to the alternative of method 2.

A further alternative according to the patent uses the presently common shade guides in linear sample configuration and supplements the same along the linear and/or vertical of this configuration. The optically effective and previously discussed “layering phenomenon”, which is created by means of and with increasing drying time and wherein, due to the clouding, the enamel layer places itself over the dentin like a bright mist and moves its (dentin) coloring into the background, can also be realized according to the patent within the patterns, in that this effect is imitated and simulated by means of material composition and/or material layering and/or painting, for example. The design of the (tooth) color palette according to the invention is not bound to a specific form of the color pattern, for example. According to the patent there is, furthermore, no limitation with reference to the arrangement and the material of the samples, sample size, sample amount/extent, etc. The invention does not define whether the samples are isolated, are located substantially independent from one another or from the carrying palette, for example, or whether they are fixedly bound to one another or on a carrier unit (e.g. palette, packaging, etc.) or how they are stored. It is also not limiting, how and in what form the patterns are held on the palette or on one another or how they can be removed therefrom. An example of a storage case for such samples is characterized according to the claims in that it organizes a system, for example in the form of time-dependent patterns according to the (tooth) color spaces and organizes the same or the patterns according to the time periods or points of time and allows and maintains this system in that the patterns or (tooth) color spaces (partial) palettes are maintained in the storage in a stable manner according to the system. This can be made possibly by means of button systems, male parts-female parts systems, zip fasteners, attachments, etc.

A general color palette according to the claims is characterized in that the color samples or color sample groups are assigned or can be assigned to the corresponding points of time and/or to the corresponding time within this process (e.g. water absorption, liquid absorption, release, condition change, etc.). An alternative according to the claims is that a process-related substantial or material-specific condition and/or a process-describing point of time is assigned or can be assigned to the sample. The method for using color patterns uses the determined pattern data (reference data, visually acquired) or patterns (reference patterns/samples) for an information acquisition by means of a current visual pattern determination, wherein, in terms of color, the pattern is to subjectively come close to the material or substance, which is to be evaluated. According to the claims, the patterns should be associated with the process points of time or periods of time and/or with the process-related substance or material conditions, for which this coloring prevailed. A metrical analysis of these newly established color pattern palettes or common (tooth) color patterns or of materials with the same identification or the same color metrically via the equipment in a metrical manner (e.g. color measuring equipment, light transmitter-light receiver system etc.), which are also used at the tooth, allows for comparisons. Data thus become comparable. If, for example, in the wake of the determination of the time of death, etc., patterns are chosen which visually achieve substantially the same result with regard to color as the tooth appearance and if these are compared thereto for the measurement at the same points of time, the correction factor, which describes the difference between visual sensational selection and objective measurement, is to be determined or the correction factor for each color measuring value is to be individually determined. Measuring values and visual determinations become comparable by means of the description of the patterns in data or value form. Measuring values can be converted or coded into indication, identification and/or information content of the color patterns and these can be converted or coded into measuring data, for example color measured values, data, or the like. Additionally, according to the patent, it becomes possible to illustrate the visual determination in the form of a diagram based on numbers or data, for example. Communication becomes possible between a color specialist and a forensic physician and/or a dentist, for example.

A tooth, which has run through a liquid release process, e.g., from its liquid-saturated condition to its air-dried condition to its equilibration weight, for example, or a portion of this process and/or the opposite process or partial process and/or which was metrically accompanied, e.g., in its spectral composition of the light hitting the sensors or of the electromagnetic radiation and/or in its courses of radiation and/or via patterns, can be identified by means of the measured data and/or determined patterns, when it again runs through the same process or partial process and is metrically and/or visually evaluated and again detected with the same instrument and/or the same pattern and conditions. If the data and/or patterns of the process repetition or the data and/or patterns approach the data and/or the patterns of the old detection at a corresponding point of time or at corresponding points of time, it is to be assumed that it is the same tooth, a tooth from the same individual, or the same individual, who carried or carries the teeth to be examined. The internal and external tooth structure is individual to such an extent that the directional development of the data is as unique as the tooth itself. At best, teeth from the same individual and, in particular, front teeth with the same identification, i.e., front teeth in the center or on the side, for example, canine teeth, premolars or molars have greater conformities in the liquid release and absorption processes detected in the data. The same also holds true for the pattern detection. If this color pattern detection is transferred into numbers, a comparison of the data and value level can also be used for identification purposes in relation to the time factor and the development of the data or values.

According to alternative methods, a data detection (reference data and/or reference patterns) (e.g. detection based on electromagnetic radiation and/or light and/or course of radiation and/or spectral composition and/or color pattern determination and/or comparative sample data and/or common methods for the liquid content detection and/or water content detection and/or condition detection according to presently known methods, analyses, etc.) takes place on substance(s) or substance sample(s) (e.g. calibration of the data with reference to the condition and/or water and/or liquid content, etc.), the water and/or liquid content and/or condition of which is known. This can take place in a further alternative method during and/or opposite to a process (e.g. chemical reaction, physical reaction, water absorption, water release, liquid absorption, liquid release, color-relevant, reflection-, changing light-, electromagnetic radiation-changing process, effect, air drying of a liquid-saturated or liquid-carrying substance, storage of a dry or a drier substance in liquid, etc., or the like) or of a portion of this process, whereby it is sensible that the detection of the data takes place from time to time. The calibration can take place in a more differentiated manner, the smaller the time interval. This substance can be involved in a main process or can be the only component in a main process or of the main reaction or can also be involved only within a side process, which runs in addition and which is associated with the main process, e.g. as an indicator. The data and/or patterns are correlated with the corresponding points of time or time periods and/or time-dependent substance or material conditions.

In an alternative method, only the condition and/or the liquid content and/or the water content with presently known common, presently known detecting methods or equipments is to be correlated with the data and/or pattern, detected on the basis of the reflected and/or passed light and/or electromagnetic radiation and/or the metrically and/or visually detected color. The detection of the connection between one and/or more of the conditions, which a material can assume, with presently known common condition-detecting methods or equipments with the data (and/or pattern), detected on the basis of the reflected and/or passed light and/or electromagnetic radiation and/or the metrically and/or visually detected color is not bound to a process, according to a further alternative method, but can also take place at one or more samples, the condition of which is known, without having to undergo a process. Both procedures represent alternative methods according to the claims.

In common liquid content and/or water content measuring methods (e.g. thermo-gravimetry, destructive common liquid content detection methods) one or more samples can be detected, whereby it is sensible hereby to dry these samples over differently long periods of time and/or to store dry samples in liquid over differently long periods of time or to add liquid thereto and/or to bring them into different liquid content conditions in order to subject them to detections.

At least one, but, better yet, several of as many different liquid content or liquid content data as possible or a series thereof are assigned to the data. By means of a current detection of data (e.g. based on light, electromagnetic radiation, etc.) (and/or pattern) at the “object”, which is to be examined, based on the reflecting and/or passed light and/or electromagnetic radiation and/or the metrical and/or visual color detection, a method and/or water and/or liquid content and/or moisture of a substance can thus be measured in the methods on this data basis (and/or pattern basis) and/or determined and/or a point of time of another predefined condition and/or a condition at a predefined point of time, e.g., in the future and/or in the past, or the like, can be detected, e.g., by means of standard data collection, computer programs, etc.

A measurement or detection of this light and/or of other electromagnetic radiation and the use of the data utilizable therefrom can now determine the liquid or moisture content of the substance and/or of the material and/or of the object. According thereto, the liquid or moisture content can be measured or determined solely on the basis of calorimetric detection or light reflection and/or light transmission detection and/or reflected and/or passed other electromagnetic radiation and the data resulting therefrom.

If comparative patterns are established and/or assigned, which visually or calorimetrically come close to the substance and/or material and/or object, being in a condition of a determined and/or detected liquid content and/or moisture content and/or water content, this comparative pattern is representative for this liquid content and/or moisture content and/or water content, which can now be determined with this pattern. A possible procedure is described in an exemplary manner in the following: A liquid-saturated substance and/or material and/or object is visually or colorimetrically assigned to a comparative pattern, which is present or which must be established. The substance in the form of a sample or several samples is subjected to the drying. During the drying, the water and/or liquid contents and/or moisture are detected according to common methods and are calorimetrically or visually assigned to a comparative pattern. Likewise, as a further possible alternative example, a dry substance can again absorb water and/or liquid by means of water and/or liquid storage, and this absorption is accompanied by measurement(s) of the liquid content and/or moisture and/or water content and the determination or assignment or establishment of comparative samples. With these procedural examples according to the patent, an assignment of one or of more, but better yet, as many assignments of color samples as possible to the liquid contents is to be achieved. Furthermore, the liquid content of sample rows, of samples with different liquid content can be determined according to common methodology and can visually be assigned to a comparative pattern. It is also unimportant herein which liquid methodology is used for the detection of the substance parallel to the pattern assignment. According to the claims of the patent, it is unimportant whether the sample has the same origin, the same substantial origin, whether the material is identical or similar as that, which is to be detected and evaluated and must thus possibly be in a comparable condition of the liquid content/moisture/water content, or whether this material was correspondingly processed in terms of color or whether it is a completely different substance, a completely different material with similar color effect or a color-matched substance or a color-matched material. The adaptation in terms of color of dental prosthesis at human dentures in the dental practice is not carried out with natural tooth patterns but with artificial or ceramic tooth color patterns. According to the invention, the liquid content and/or moisture content can now solely be determined by means of the (color) samples. A liquid content and/or moisture and/or water content or the material carrying a defined liquid content and/or moisture and/or water content can now be assigned, in a substance-specific manner, to each pattern. The detection of the liquid content by means of comparative patterns, which, for the purpose of determining the liquid content, must come close to the substances of a defined liquid content in terms of color, is thus made possible.

Detections of water, liquid content, moisture, condition, or the like can now be carried out free of destruction without taking a sample. Depending on the equipment, the localized liquid measurement often takes longer than one second and is thus advantageous for time-economical reasons. Advantages of these methods are the localized destruction-free moisture content measurement, water content measurement, liquid content measurement via portable measuring instruments, the accuracy and precision (depending on the requirement and the used instrument and the effort to far below +/−0.01%) of the results, the simple handling (one push of a button is sufficient and the result can be read and/or compared with a standard diagram and/or calculated, or the like, etc.). In a light transmitter-light receiver system, which can also detect the course of radiation, it is advantageous that for each tooth and/or substance and/or each sample and their characteristics an individual choice can be made, as to whether only the composition of the spectrum and/or the detected intensities and/or the course of radiation can or should be used. It would be sensible, for example, that, the older the teeth and the less liquid they carry, the sooner the spectrum and the course of radiation should be measured.

Presently, standard diagrams of colors or water content alone have been considered to be too insufficiently differentiable for these methods to be able to stand on their own so that, until now, it was only possible to make statements regarding the statement-strengthening connection, liquid content, and color value and by their common monitoring and evaluation. By simulation, the accuracy of the informational value can be enormously increased so that exclusively one or more data, e.g., color-describing values and/or of the spectral composition of the light and/or of electromagnetic radiation and/or to its and/or their course of radiation and/or even of the liquid content and/or of the water content are possible and/or can be highly accurately detected and used according to the claims. There is a demand for the use in the most different commercial areas for these measurements of the liquid content and/or of the moisture degree and/or of the water content and/or of the condition and/or of the determination of the point of time and/or of the condition determination and/or prognoses and/or estimations and/or reconstructions of points of time and/or the condition and/or the liquid content, etc. according to the claims by means of data detection via light and/or electromagnetic radiation and/or course of radiation and/or spectral composition: examples of use are control, drive, regulation and monitoring of production cycles, e.g., in dental, construction, textile, color, printing, paper, food, tobacco, agricultural, electronic, electro, chemical industry, pharmaceutics, agriculture, raw material processing, reprocessing (e.g. plastics, bricks, stones, pulps, leather, wood, soil, sand, concrete) industry, threshold value determination and threshold value monitoring.

When the claims and/or the description thus mentions samples or object or material or subject or substance or structure, they, of course, refer to and/or sometimes synonymously identify the object and/or material and/or subject and/or substance and/or structure and/or building material and/or processing material and/or structure and/or grit and/or bulk material and/or powder and/or chemicals and/or natural substances and/or textiles and/or building material and/or paper and/or gases and/or gaseous and/or solid and/or liquid substance and/or a sample thereof, etc., or the like. The meaning or relevance of none of these aforementioned words is limited and/or limited to a certain area.

Measurements on retaining walls, dams, and structures, soils, evaluation of material processes, e.g. thickening process or moisture content, drying process, etc. of bricks, lime sand bricks, volcanic tuff, sandstone, granite, marble, concrete, plaster, mortar, porcelain, ceramic, grit, bulk material (sand, gravel), masonry survey, liquid determination of rock or grounds in geology, questions regarding the coloring, liquid content, drying process in textile industry, substantial relevant processes (plaster setting, moisture, ceramic processing, duration of the fire, the processing, etc.) in the dental technology, etc.

Measurements, control, monitoring even from a distance (e.g. camera, image recordation, laser, etc.), e.g. of conditions, procedures, reactions, processes, etc., which influence reflected and/or passed electromagnetic radiation and/or light and/or spectral composition and/or course of radiation and/or which are relevant in terms of color.

Prognoses and/or reconstructions of, e.g. when a corresponding process condition is reached or was reached or how the color condition and substantial condition or the situation regarding the substance will be or was at a certain point of time and/or which data and/or effects prevail here, can be achieved according to the patent. Reconstructions and/or determinations of points of time and/or substantial conditions are thus also possible according to the patent. The planning of a chronological but also substantial type becomes possible. Processes can be accelerated, for example by means of more highly concentrated reaction partners, heat exposure, catalysts, or the like, and this procedure can be quantified. Chronological estimations become possible. Process planning, process monitoring and/or controlling and/or the establishment of the current condition and prognoses or reconstructions of points of time, color conditions, conditions of the liquid content, of other substantial conditions, also in reference to time, and the like, have been made possible according to the patent.

Examples are the liquid absorption or liquid release to materials. The color condition or liquid content of other points of time of the liquid absorption or release methods and also, for example, the condition of drying by a dentist can be reconstructed (dental color reconstruction) by means of metrical or visual detection(s) from colors, or, correspondingly, metrically from data of the dried object with the knowledge of the drying time. The determination of the post-mortem period of the corpse can also occur, according to method 1 and/or 2 and/or one or more of the methods according to the claims by means of e.g., bleach and/or liquid absorption and/or drying processes and/or other condition changes on the clothing and/or the skin and/or the hair and/or the bones and/or the clothing, or the like, of the deceased, instead of on the teeth.

The control, the monitoring and planning of teeth bleaching processes, of how long bleaching must occur, for example, to achieve a correspondingly desired result, are to be made possible. Likewise, the bleaching process can be planned, controlled, etc., in the paper industry. Further examples are: determination of the degree of decomposition also via the time factor and the color in forestry, decomposition of the corpse by means of, for example, the skin color or the coloring of postmortem lividity in the area of forensics, coloring progress control and planning in the textile industry, browning planning and process control for skin exposure in the beauty-related industry and in medical science, laser light control in medical science with regard to the problem of how long a laser, for example, may (still) be on the skin without causing unintentional damages, blackening of X-ray indicators (film detectors) by X-ray for the determination of the application rate of radiation and of the time, during which the medical personnel or the personnel of nuclear power plants, for example, may still stay in the vicinity of radiation, so as not to exceed the corresponding threshold values of the yearly dose, the extent, e.g., of the patina development on metals for the purpose of determination of the object in archaeology, changes by heat treatment, chemical reactions, biochemical and physiological test, indicator reactions, etc., determination of the degree of ripeness of fruits and vegetables and the estimation thereof according to the storage conditions, for example, how long they will stay fresh or when they will have reached the corresponding ripeness (e.g. determination of time of ripeness, of the actual condition, planning, etc.).

Likewise, a prognosis can also be made regarding the changes of concentration or concentration conditions can also be reconstructed with reference to the time, e.g., by means of the detection. Planning and prognoses, for example, are also made possible here.

Areas and examples of use can be found wherever substances/materials exist, which are able to absorb and/or release water and/or liquid or which contain liquid and/or which can assume and/or change a condition. A destruction-free measurement on retaining walls, structures, nuclear power constructions, liquid of the soil, e.g., for geological or meteorological purposes, for the purpose of surveying a house, a room and soil, wood evaluation in forestry or in architecture, for assessing processes during which liquids are released or absorbed (e.g. hardening of plaster, concrete, mortar, ripening, etc.).

A color pattern palette according to the claims is characterized in that corresponding liquid contents or moisture contents can be assigned or are assigned to the color patterns or a color pattern group in a substance-specific manner and/or other condition forms (e.g. material mixtures, concentrations, chemical compositions, etc.). It is possible, specifically for substantial conditions or material condition possibilities, to establish or assign thereto patterns, in order to determine the condition of the substance, material, object or several thereof, solely by means of the patterns. The assignment of the condition to the described data or of the color patterns to a reference data or exemplary data bank or exemplary data or pattern pool requires a parallel detection by means of at least one common detection method and at least one data detection by means of instruments and/or methods according to the claims.

A color pattern palette according to the claims is characterized in that codes for the corresponding condition, for example, wherein the colors of pattern and substance visually correspond or come close to one another or data and/or information describing a corresponding condition are assigned to the patterns. A possible light transmitter-light receiver (sensor) system, which is suitable for one or more the methods is characterized by: at least one or more light transmitters and one or more light receivers and/or sensors and/or detectors. The data processing connected downstream, which includes the topographical position of the light transmitters and/or of the light receivers (e.g. sensors, detectors, photocell, camera, color measuring instrument etc.) into the calculation, can thus, in addition to the calorimetric and/or spectral light processing and/or processing of the electromagnetic radiation, also include the course of radiation and its process-related change and/or can thus, at least if necessary, also detect statements regarding the changes of the course of radiation with the course of the process. Light, refracted with the process in a modified manner, generates different data or value levels by means of different intensities, which act on the sensor in the different regions of the detection space. The light detection can take place via sensors, for example, in the entire space or can amount to only a portion of the detection space.

As an instrument, the alternative methods use at least one a sensor and/or a camera and/or a detector and/or image recording and/or color measuring equipment and/or light transmitter and/or generator of electromagnetic radiation and/or pattern with corresponding processing of the information. The use of a neuronal network (modularly designed calculation models according to the principle of the biological example with the characteristic of learning aptitude), which forms the basis of an alternative claim, is proposed for the detection and/or processing of the data and/or intelligent image processing, possibly also with the inclusion of simulation factors. According thereto, the system is to optimize by itself and/or cohesively the detection method and calculation method and/or the influence of factors (humidity, temperature of the ambient air, the climate in the mouth, body temperature, saliva consistency, composition of the saliva, etc.) and is to aid in the increase of the precision of the determination of the time of death and/or of the determination of the post-mortem period of the corpse and/or the tooth color construction and/or of determination of time and/or liquid content and/or water content and/or condition detection and/or reconstruction, etc. The neuronal network is also to be used, e.g., for process planning, for prognoses, reconstruction, measurements, etc. on materials and/or is also to incorporate material-specific characteristics.

The system or the detection machine and/or the instrument for the measurement at a tooth and/or material can be portable (e.g. as portable measuring instrument) and/or stationary. It is thus possible, within an alternative method, that the condition and/or the time of death, or the like, is determined by a direct measurement on-site on the basis of the reference data.

One or more of the methods can also be carried out exclusively via, e.g., one or more sensors, detectors, a camera system and/or image recording and/or image processing and corresponding data processing, etc. The advantage of the detection of a visual information or of the topographical resolution lies in that a section can be selected and/or retrieved, individually for the tooth and/or the substance and/or the material, for example by means of a computer program and/or a searching system and/or via the neuronal net and/or the factory setting by the manufacturer and/or the adjustment of the operator and/or of the user of such system, which is (better) suitable for the evaluation and for the method purpose. One or more sections and the sizes thereof can be selected in such a manner that there is at least one possibility for the optimization of the data detection or evaluation on the basis of the data.

In particular, in one or more of the methods of this application is may sometimes become necessary to detect several and/or as many samples as possible, for example by means of a measuring process, at the same time or one after the other and/or in an automated manner and to always detect the samples at the same location, for example along a measuring row. The location of the same measuring surface for, e.g., the colorimetric detection and/or detection of the course of radiation and/or spectral composition of light reflected and/or let through by the object and/or reflected and/or let through electromagnetic radiation etc., or the like, on, e.g., complex irregularly, or even individually curved surfaces, for example at the tooth, are sensible. Such a detection system can be used for one or more of the other methods, e.g., in particular, for the determination of the time of death and/or for the determination of the time and/or for the determination of effects of artificial age and/or the substance-specific conversion of water and/or liquid content measuring values and/or condition measuring values into data (e.g. condition-specific and/or substance-specific and/or device-specific gaging and/or calibration, etc., or the like) detected according to the claims, and/or the identification of objects and/or creatures by means of process-related condition changes, water content changes, liquid content changes, water content and/or liquid content and/or moisture measurement and/or condition measurement and/or detection etc., and the like, can be realized, optimized and/or carried out under absolute accuracy by means of a detection system and/or detection machines and/or can be made possible in large amounts of objects (e.g. teeth, materials, objects, samples, etc.).

One of this automization and/or rationalization of, e.g., the liquid content analysis and/or the water content analysis and/or the degree of moisture analysis and/or condition analysis (e.g. on the basis of the light and/or of the electromagnetic radiation, etc., or the like), e.g., via air drying and/or accelerated drying via the heating, liquid absorption, etc. is made possible as an alternative according to the claims (inclusion of the process-related time factor). An accelerated drying has the advantage that the process is less time-intensive, however, it also generates less accurate results.

For the benefit of the accurate detection of data, the acquisition of the data is favored under normally occurring drying conditions for the use of the determination of the time of death, in particular for method 1.

Data records from the different detection methods can thus be pooled, e.g. according to method 2. It thus becomes possible to also detect or measure the degree of moisture or the liquid content calorimetrically or by means of the above-mentioned analysis of the light and/or of the electromagnetic radiation. These substance-specific conversions of liquid content measuring values, for example in color measuring values and/or by data supplied by corresponding detection instruments, make it possible to detect the same, even in larger amounts, even of different substance samples, successively and/or simultaneously. A relation to the process-related time factor and/or the determination of the time of death are optimized in this manner.

According to the claims, a sensible arrangement of the samples and/or of the teeth is proposed next to one another and/or linearly and/or in a circle (FIG. 12) and/or on a surface having any shape (exemplary possibility FIG. 5a) (e.g. square, rectangular, circular, etc.) and/or in the space, for example. Each sample can be separated from the remaining samples or from several of the remaining samples by a wall or separating wall (2) and/or each sample is located in a container FIG. 5a (3) and FIG. 5b, separated from the remaining samples or from several of the remaining samples. All of the samples can furthermore be separated from the ambient air by a capping according to FIG. 5b (4). The storage of each sample (e.g. teeth, tooth, substance, material, sample, etc.) is thus ensured independent on the influence of the surrounding ambient air. Furthermore, the samples are thus independent from one another and, due to an isolation of the samples by the wall (2), it becomes possible to treat each sample differently and to subject each sample to a different climate or microclimate. For this purpose, a heating unit (9) and/or a supply device (7) (e.g. a hose, pipe, supply, etc.) of corresponding air or liquid is provided for achieving wanted or desired air climate or liquid amounts (e.g. warm or cold air, temperature of air and liquid, air pressure, liquid amount, type of liquid, consistency, specification of the humidity, etc.) is required according to the claims. That, which is present in amounts that are too high or that which is to be removed (e.g. liquid, humidity, etc.) is withdrawn from the container (regulation of the process) via the discharging unit (5) and/or via the supply device (7) itself. All of the containers and/or a main container or allocation units (1, 3) can thus contain an inlet to and/or an outlet for liquids. All of the containers or allocation units are deep-seated with one another and/or with a base plate or can be attached at a predefined location and with accurate repositionability. The regulation of one or more of the conditions or simulations according to the invention are controlled, e.g., via a control circuit. For this purpose, a qualified sensor technology is required for a corresponding detection of corresponding condition(s) within and/or outside of a container and/or allocation unit and/or of the machine. According to the invention, within and/or outside of the spaces or units, the climate of the liquid content, the liquid consistency, etc., can be determined. Simulations of ambient air conditions, conditions to which a sample must be subjected (e.g. determination of the time of death), for example, or, e.g., which a sample is subjected according to experience (e.g. artificial aging) are possible. The inlet and outlet (e.g. air, liquid, etc.), the heating processes and/or the capping and/or decapping and/or the relocation of the measuring surface can take place by means of motors, mechanically and/or manually and/or via a program and/or automatically and/or via corresponding specifications (e.g. periods of time, points of time of measurements, liquid amount, air temperature, humidity, components of the air, air composition, foreign matters in the air and liquid, liquid composition, control processes, etc.). The maintaining of a position and/or of the relation between measuring object (10) (e.g. sample, tooth, etc.) and detection instrument (e.g. camera, sensor, detector, measuring head of the measuring equipment, etc.) (12) is realized as an alternative according to the claims and/or via the location and/or relocation of the measuring surface by the positioning of the sample below the accommodation area or instrument (42) and/or the same in relation to the sample as further alternative methods. Maintained relations are realized, e.g., by means of maintaining the contact of color measuring equipments, which operate in contact mode, e.g., also via fiberglass (FIG. 6) or via a row of detection units (FIG. 9, (17)) or in color measuring equipments, which operate in non-contact mode, camera systems, etc. in a fixed reaction to the object or to the measuring surface at the object. See, for example, FIG. 8 and FIG. 9 (16) thereto. In the case of the equipment, which operates in non-contact without permanently maintaining the relation between object and, e.g., detector, detection unit, sensors, etc., it is proposed, according to the claims, that the optimal relation (e.g. distance, measuring surface, etc.) of the detection region to the object or the sample takes place either with a program-based automatic positioning of the sample or of the object and/or the relation takes place via the program-based positioning of the technical detection unit and/or of the detection region or are purely optically realized via sensors and via the focusing, e.g. automatic or manual focusing, so that the measuring surface has an optimal distance to the detection unit for the purpose of an optimal measurement.

According to the claims, the positioning and repositioning can occur, e.g., via a motor or micromotor and/or via manually guided measuring equipment and/or via the sample(s), whereby the guide and/or the end position can be determined, e.g., by the program, (micro) motor, precision attachment, ball bearing, drive definition, by end stop positions, by ball or groove engagement, positive engagement in negative depression, by telescopic support with precision attachment and stop, etc. The samples, e.g., can thus be guided in relation to the instrument, e.g., lengthwise and crosswise (FIG. 5a) and/or on e circular path (FIG. 12) (e.g. by aligning guide of the sample basis or of the instrument, rotation of the sample basis) and/or the instrument is correspondingly guided in relation to the samples.

Furthermore, one or more of the detecting equipments or of the instruments can also be positioned and repositioned in relation to the object or the sample, or vice versa, in that the form of the same measuring surface on the object, which is determined during initial measurement or which prevailed by chance (e.g. internal and/or external geometry, or the like) was detected (e.g. scanning, laser, camera, inner structure, etc.) and the same topographical position is found for the repeated measuring via the “shape” (sensory alternative). A further alternative method uses, e.g., predefined adjustment positions between the measuring instrument and the sample-carrying unit through the computer program and/or the predefined dimensioning of the running gear and/or of the positioning equipment, which moves samples and/or measuring instruments, etc. (mechanically-determined alternative).

During the positioning and/or repositioning of the samples, these are linearly arranged, e.g., on one basis, it is also possible via a rail on which the sample(s) is/are moved and/or the measuring instrument(s) and/or the measuring unit(s) is/are moved in relation to the sample(s). If samples are arranged above a surface (FIG. 5a), at least two rails (41), which are arranged vertically to one another and which are displaceable against one another, are necessary to displace and reposition the sample(s) and/or instrument(s) or detection unit(s) (42) in relation to one another. If samples are arranged in a space, at least three rails must be spatially arranged, and also be displaceable against one another vertically in all directions of the space for the relative displacement and repositioning of the sample(s) to the measuring instrument(s) or the measuring unit(s).

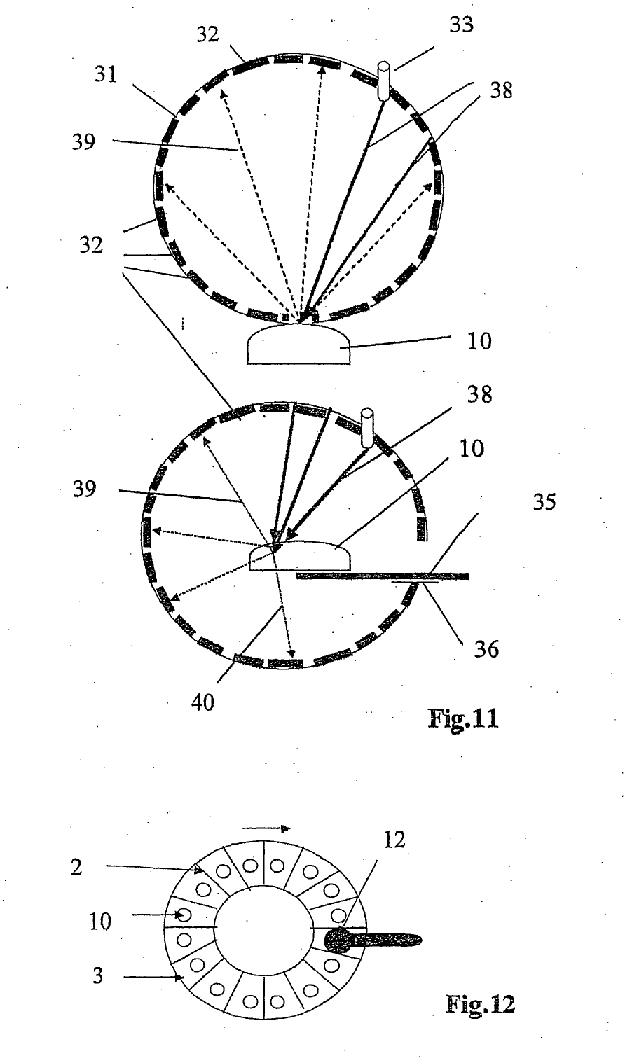

In a further alternative method, a sample according to FIG. 11 (10) or several samples can be positioned in front of a measuring space (FIG. 11, top) or in a measuring space (FIG. 11, bottom). The walls of the measuring space illustrated herein in a spherical manner, for example, consist of one or more detection units and/or detection instruments (32) and one or more light transmitters or units emitting electromagnetic radiation (31 and/or 33). The radiation (38) hitting the object is either reflected in a diffuse manner (remission) or reflected in a directed manner (39) and/or passed by the object (40). If one or more of the objects is/are measured within a measuring space, the same can be transported into the measuring space via a carrier system (35) via a rail (35, 36), for example. The smaller the detection units and/or the more detection units and/or detectors and/or sensors and/or detection instruments and/or light transmitters and/or units emitting electromagnetic radiation and/or the greater—to a certain degree—the surface of the measuring space, which is lined with one or more of these aforementioned elements, the more accurately a statement can be made or a differentiable measuring result can be acquired, due to a topographical analysis of the localization of the radiation and/or in relation to the emitted radiation and/or of the course of radiation or its process-related change. Each sample can be held in situ at least to the basis, to which it is to be mounted, via a fixing mass (6). Such a mass can be, e.g., viscous, soft, liquid, etc., and can, over time, be or become hard, stable to deformation, adhesive, etc., by means of chemical cross-linking or by physical setting, etc. With such a process by means of the walls of the space or container or of a detection unit or by means of a provided negative and/or positive relief, which, in turn, stands in a fixed relation with the detection unit or which can assume the same and which makes it possible to completely or partially absorb the mass, in order to enter into a bond, a context, a key-lock-similar positive-negative transition thereof, etc. (see explanations regarding the fixing mass). According to the claims, as an invention alternative, a measuring unit can be set up above each sample, for example in the form of a fiberglass end piece (FIG. 6 (8a)), and/or photocells and/or sensors and/or cameras and/or color measuring instruments and/or portions thereof, etc. (FIG. 6, FIG. 9 (16, 17)), without them changing their relation to the objects (e.g. samples, tooth, etc.) during a measuring row. In this alternative, the fiberglass cable (8) is held with the sample (10) via a stabilizing element (11) of the unit on the one hand, and, on the other hand, it is connected with the equipment or system (12) at the measuring side and/or a system (14) with measuring instruments (17) operating in contact mode is in contact with the probe with its measuring head or, in the case of a sensor and/or measuring instrument and/or a camera, or the like, operating in non-contact mode, in a fixed relation with the sample FIG. 9 (16) as further alternative of the invention. In this case, more than one or, better yet, several or many measuring units (e.g. transmitter-receiver systems of electromagnetic radiation, light, cameras, photocells, fiberglass cables, color measuring devices, etc.) are necessary. If a fiberglass cable is used, a camera or a sensor or color measuring device, or color measuring equipment may be sufficient, if between fiberglass cable (16) and one or more thereof, a “distributor” or “deflector” (only an exemplary illustrating possibility and can also be realized in a completely different manner) enables the distribution and the position of points of light and/or of other electromagnetic radiation (e.g. according to FIGS. 6 (12a) and 7) and/or image formation. This electromagnetic radiation and/or this light is transmitted (18) on, e.g., a mirror or magnet system, or the like (19), which, e.g., by means of a rotational device (21) can be directed to “inlets/outlets”, which are open and/or closed and/or can open and/or close, or to the ends of the fiberglass cable, which are open and continuous herein (46) or which are illustrated in a closed and impermeable manner (47). Separating walls or separating protrusions (20) separate the inlets/outlets from one another, so that passage fields are formed therebetween. A camera and/or a corresponding detection instrument also suffices, which establishes a (complete) image of the sample (e.g. teeth), which are used for the evaluation (FIG. 8), e.g. for one or more methods according to the claims used for the determination of the time of death, and the like or which acquires image information, whereby the possibility exists herein to isolate the regions used for the evaluation from the complete image by means of an image-processing program, e.g., via marking or determination of the section or (15) and that this region/those region(s) and isolated region(s) and/or the reflected and/or passed electromagnetic radiation and/or light are analyzed in terms of color and supplies/supply data (FIG. 10). In the alternative of a visual detection, the size, expansion, number, etc., e.g., of sections or measuring surfaces can be determined by one or more samples in an image program, or the like. The image information can also be used in that, absolutely or relatively to other image locations, the color (via, e.g. RGB-system, the grey or color stages, intensities, etc.) or radiation pattern and/or spectral composition and/or course of radiation etc. (e.g. light, electromagnetic radiation, etc.) is analyzed. The device according to the claims and/or the system according to the claims provides that the process operations, an automation and/or the courses of operation run according to predefined programs and/or include such a program. According to a predefined program, for example for the termination of the time of death, the sample can automatically be brought into relation to the measuring instrument, or, after the sample was placed by hand, the measurement or measurements can be carried out, liquid can flow thereto or can be manually added, the liquid can again escape correspondingly, measuring rows with predefined points of time and periods of time at which or after which the measurements are to take place to the point of time or the time interval at which the measuring results come close to or are identical to those at the point of time of the first measurement. Furthermore, it is proposed as an alternative according to the patent that an automatic operation transmits a signal at that point of time or that the system shuts down. An alternative according to the claims establishes a protocol including the time of death or the time period prior to the first measurement from which the time of death results, by means of the measuring values and the time periods, at which they had been measured.

In this manner, the conversion of substances described in the same above-mentioned patent application, the water contents, liquid content values, the degree of moisture, the conditions of which are known, can also be converted into data according to the claims (calibration, gaging). For this purpose, at least this equipment or this system or a detection unit (3) must additionally have, for example, a further common and/or known instrument for detecting conditions, e.g. a weighing unit (9) or a conductivity measuring unit etc., or the like. According to the claims, the same equipment or the same system is now able to enable, in an isochronal or almost isochronal or parallel manner or within the (predefined) chronological tolerance, detections by means of common methods (thermogravimetry, gravimetry, NIR spectroscopy, conductivity measurements etc.) and detection of reflected and/or passed light and/or electromagnetic radiation, the composition and/or course of radiation and/or color and/or other data acquisition possibilities thereof at the substance.

In doing so, the same instrument or the same detection system can, in a substance-specific manner, connect data, which are based on detections, by means of common methods and with data from the detection of detections according to the claims (e.g. based on light and/or electromagnetic radiation, composition, course of radiation, etc.), see above.

The same detection system can now measure the degree of moisture and/or of the liquid content and/or of the water content in a substance-specific manner, due to exclusive detections of the color and/or of the light and/or electromagnetic radiation reflected and/or let through by the substance and/or its and/or their composition and/or its and/or their course of radiation. If this detection corresponds to or is brought into correspondence with other equipments, which operate on the basis of light and/or other electromagnetic radiation, the degree of moisture and/or the liquid content and/or the water content can then also be measured therewith, or the detection system can be gaged thereon and can establish, in a substance-specific manner, the reference data gaging and calibration and one or more of the relationships of data according to one or more of the claims. Likewise, an exclusive detection possibility is given, by means of the herein assembled common methods (for example thermogravimetry, gravimetry, infrared, conductivity measurements, etc.), in particular for one or more of the methods according to the claims.

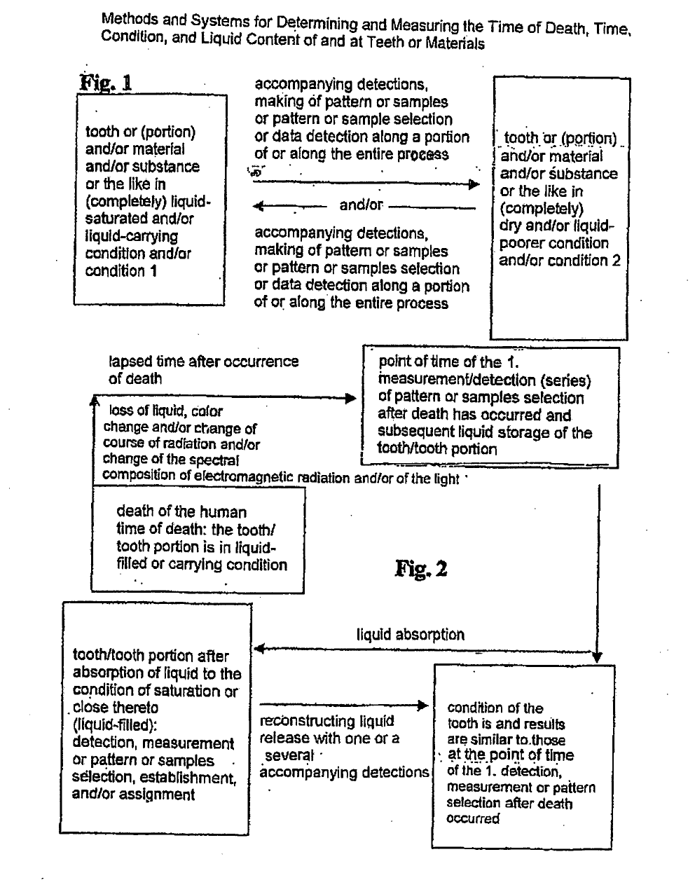

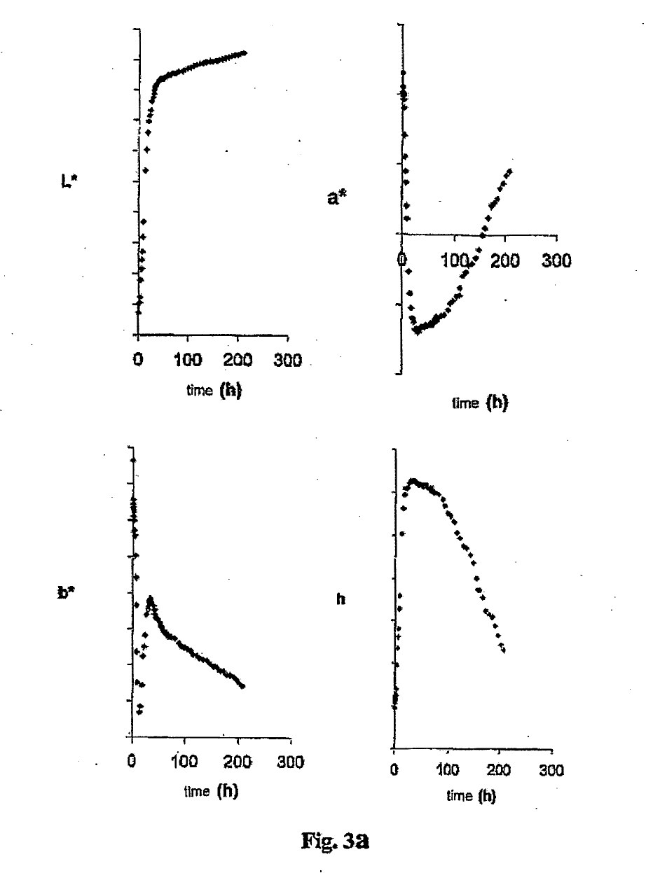

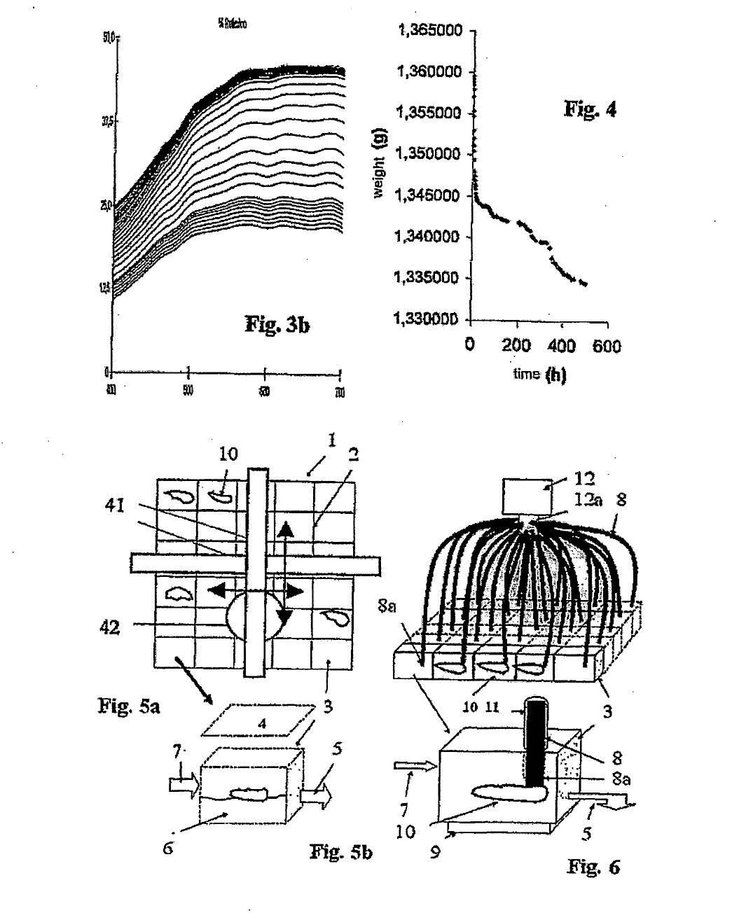

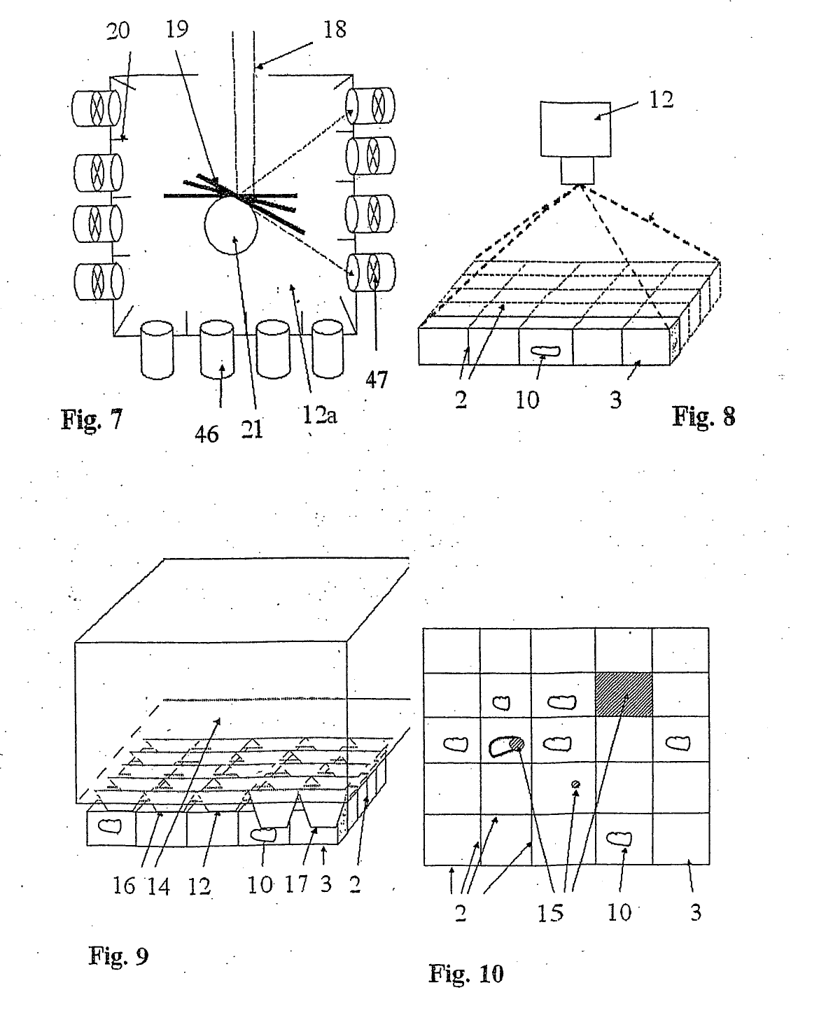

For clarification purposes, FIG. 1 schematically illustrates a possible process in an exemplary manner according to method 2. For clarification purposes, FIG. 2 schematically illustrates a possible process in an exemplary manner according to method 1. For clarification purposes, FIG. 3a illustrates a possible development of the color measuring parameters or coloring measuring values, in the instant case, in an exemplary manner, of the L*, a*, b*, h* values of the CIELAB and CIELCH system of 1976. For clarification purposes, FIG. 3b illustrates a possible development of the spectral curve. The reflection (%) increases with an increasing drying time. For clarification purposes, FIG. 4 illustrates a possible development of the liquid content decrease in the exemplary case of the gravimetrically detected liquid content or of the absolute weight of a tooth. The weight of the object or of the tooth decreases with an increasing drying time. FIG. 5a illustrates an aereal arrangement of the detection units (FIG. 5b). FIG. 6 shows an instrument, which operates in contact mode via fiberglass cable (8), whereby a possible distribution of, e.g., application light and/or detection light occurs via a distribution system (12a) and (FIG. 7). The instrument in FIG. 8 operates without contact to the object. A region (measuring surface), which is used for the detection, can hereby be predefined or determined according to FIG. 10 (15). The detection instruments in FIG. 9 show the possibility (equipment alternatives thereof or different equipments) of a contact measurement (17) or of a non-contact measurement (16). FIG. 11 shows a detection space, whereby the sample is detected outside (FIG. 11, top) or within this space (11, bottom).

According to the claims, the dimensioning and the technical degree of the embodiment, and the number of elements of this system are left open. According to the claims, it could also be possible that a simulation region or the detection system or the detection unit is, e.g., microscopically small or also has the size of a room and that many independent detection equipments, operating in an isolated manner and/or being in contact with a central processing unit (e.g. computer, processor, etc.), are located in and/or outside this space and/or have their connection to the samples located inside of the space (e.g. fiberglass cable, plate and handle of an externally located weighing unit, cable, probe for conductivity measurement, etc.) or that devices, e.g. a glass panel, permit the detection means (e.g. camera, sensor, etc.) to conduct measurements on the samples according to their capability, via non-contact. The spaces can also be climate chambers, climate cabinets, etc., for example, which fulfill the above-mentioned conditions. Detectors, sensors, or the like can also have the size of micrometers or nanometers or detection units can assume the size of entire equipments or of instruments or of measuring systems, or can be measuring equipments or of measuring instruments, or the like.

If radiation and/or spectral composition and/or intensity and/or courses of radiation of the light and/or of the electromagnetic radiation and/or of the color of the object reflected through an object and/or by an object is detected, this can occur in an absolute manner and/or in relation to the original radiation, or the like, according to the claims. If the output radiation is unknown, it must also be detected in the case of the corresponding alternative method.

A software determines and/or the user can adjust whether and which of the sample(s) and/or in which order and/or chronological sequence and/or according to which time intervals and/or under which simulation and/or under which conditions (e.g. temperature, air pressure, humidity, etc., or the like) are detected. This software can be used and/or modified by the manufacturer (factory setting) and/or by the user and/or individually according to the requirements. For the use on the concrete object, which is to be examined and/or to be evaluated, information regarding the object (e.g. type of object, material, surface composition, grain size, chemical composition, etc.) can be input via the software. With such a corresponding consideration, an increase of the measuring accuracy can be achieved during a detection via the reference data, which were established in consideration of one or more of such factors.

An instrument, which detects other data or which detects on a data level, which differs from the detection system and/or from the detection machine, can be calibrated or gaged by means of a software-based “conversion” of the detection system data and/or of the detection machine data into the instrument data, so that, with the use of the analysis or of a portion thereof by the detection system and/or the detection machine, the instrument itself can now measure and/or determine and/or calculate the time and/or time of death and/or liquid content and/or water content and/or moisture and/or condition of substances.

It is self-explanatory that the above-described exemplary embodiments or the described methods are only illustrative or that several possible alternative embodiments are described for this application. Other types, variations in the use, construction, shaping, act in combination of the components, number of components, etc., can easily be diversified or designed by an expert or person of skill in the art, who has knowledge of this application which represent the principles of the invention and which are to fall within the scope of protection thereof. It is also possible, with the aid of micro-technical and/or nano-technical components and/or other methods and/or instruments and/or equipments, which already correspond to the state of the art and/or which will be newly developed in the future, to fulfill one or more of the method purposes and/or which have corresponding method characteristics in principle, which are thus also to fall under the protection of this application.

Each method according to the invention is not limited in its location, arrangement, number and connection of the method steps, method portions or method components, and in the (technical) means used therefor. The methods according to the invention also do not have any limitation in the type, choice, quantity and the number of the means and/or of the material for the realization of the data-processing/data-comparing method steps and of those, which are used, and also in the choice and the type of the objects, materials, substances, and equipments, which are used or which are to be produced.

When the claims and the description mention reflected and/or passed light, the light originally emanates from a natural (direct and/or indirect sunlight, biological light, self illuminated object) and/or artificial light sources (LED, laser, lamp, luminary, etc.) and is used either continuously or in a temporally limited manner and/or as a flash of light, whereby the object or the tooth is directly and/or indirectly subjected to the light. When the patent claims and/or the description mention the word tooth, they refer either to a tooth or several (more than one tooth) of teeth.

The best way to acquire data, which can be used according to the claims, takes place via the detection of spectral composition and via the course of radiation of electromagnetic radiation, in particular of light, in the visible and/or invisible spectral range, whereby the object or tooth is illuminated and/or lighted and/or supplied with energy. For clarification purposes, it is to be mentioned once again that the tooth and/or substance and/or “object”, which is to be examined, of the (renewed) detection(s) of the data, mentioned in claim 2, is not the or one of those (i.e. not the same sample), which were measured during the reference data detection, but that the substance or the object are of the same type or similar. However, it is also possible according to the claims, even though it is not very sensible, to use the same sample (tooth and/or substance and/or “object”) for reference data and for data acquisition during specific application.

When If the patent claims and/or the description mention “natural” tooth, they refer to a living and/or dead tooth of a dead and/or living “living being” (e.g. person, human being, animal, individual, etc.), which is given to its carrier or living being by nature. When the patent claims and/or the description mention an “artificial” tooth are referred to dental and/or dental-technical activities and/or the used materials (e.g. crowns, bridges, filling, prostheses, plastics, ceramics, metal, etc.). For clarification purposes, FIGS. 1 and 2 schematically represent some possible processes, only in an exemplary manner.

1. A method for the purpose of determining the time of death comprising the steps:
a) First postmortem recording(s) or acquisition(s) of the condition of one and/or more artificial and/or natural teeth by means of suitable instrument for the purpose of acquiring data and registration of the when-data (time of acquisition);
b) Liquid storage of the tooth by simulation, whereby, with increasing fluid absorption, the tooth finds the data, which data the tooth had during the life time of the living being;
c) Subsequent successive drying of the tooth by simulation and accompanied by acquisition(s) by means of an instrument suitable for acquiring data, to the point, at which the data equate the data of the first postmortem measurement or at least match the data in such a manner that they are within a predefined range of tolerance; and
d) Determining the time of death by calculation back from the point of time of the first postmortem acquisition(s) back into the past by means of the drying time detected in step c).

2. The method for the purpose of determining the time of death and/or determining the post-mortem period of the corpse and/or the measurement and/or detection of a point of time and/or time period and/or condition and/or a liquid content and/or water content at the tooth and/or of a tooth and/or at the substance and/or of a substance comprising the (preceding) detection (reference data acquisition) of one or more possible fluid contents and/or water contents and/or conditions, which can be assumed by the teeth and/or the substances by means of at least one suitable instrument, which supplies reference data, whereby, if necessary, the reference data can also be detected during the process of changing the condition and/or the liquid absorption and/or liquid release and/or the water absorption and/or water release of a or one tooth and/or substance or several teeth and/or substances and/or whereby these reference data, depending on the use thereof, were related with the process-related time factor and/or the corresponding liquid content and/or water content and/or condition, whereby this reference data acquisition occurs by simulation and, in the following, in the case of (usage) of one and/or of more renewed recording(s) of one or more data for example at a tooth and/or substance and/or “object”, which is to be examined, corresponding reference data can be consulted, in order to be used for one or more of the aforementioned purposes.

3. The method according to claim 1, comprising one or more connection(s) of data and/or of color samples and/or of comparative patterns or samples and/or of the condition and/or of the liquid content and/or water content of the tooth and/or of the substance and/or of teeth and/or of substances, whereby the resulting connections in its use, now enables, for example, the detection and/or determination of a point of time and/or of a time period and/or of the liquid content and/or of the water content and/or of the condition by means of the detection of the light and/or electromagnetic radiation reflected and/or let through by the tooth and/or by the substance and/or by the object.

4. The method according to claim 1, wherein, for data acquisition, the radiation and/or radiation pattern of electromagnetic radiation and/or, particularly, of light reflected and/or diffused by and/or transmission of the tooth and/or substance and/or the teeth and/or substances is detected, in order to acquire data therefor.

5. The method according to claim 1, wherein, for the acquisition of data, light is used, which falls on the tooth and/or on the substance and/or which is applied thereon by at least one “light transmitter” and which is changed and/or reflected and/or let through by the tooth and/or the substance and which is received by at least one “light receiver” and/or the “instrument” uses light for the acquisition of data.

6. The method according to claim 1, wherein, for data detection or acquisition also or only other electromagnetic radiation than the light is used, which falls on the tooth and/or the substance and/or which is applied thereon by a “transmitter” and which is changed and/or reflected and/or let through by the tooth and/or by the substance and which is received by the “receiver” and/or the instrument uses electromagnetic radiation for the acquisition of data.

7. The method according to claim 1 wherein the light is light of the visible and/or invisible spectrum and/or of a section thereof and/or monochromatic light and/or laser light.

8. The method according to claim 1, wherein, for data detection, the composition of the radiation and/or of the direction of radiation and/or course of radiation and/or radiation intensities and/or radiation pattern serve as the basis for the data acquisition, at least additionally and/or exclusively.

9. The method according to claim 1, whereby as “instrument” at least one correspondingly suitable measuring apparatus and/or at least one light transmitter-light receiver system and/or at least one transmitter-receiver system of electromagnetic radiation and/or at least one camera and/or at least one image recording and/or at least one image processing and/or image editing and/or at least one suitable sensor and/or at least one suitable detector and/or at least one image processing and/or at least one color measuring equipment and/or at least one color sensor and/or at least one color detector and/or at least one corresponding software, in particular for the color detection and/or color processing and/or the visual (subjective) comparison by using color patterns or samples and/or comparative samples and/or at least one water and/or liquid content measuring device and/or moisture measuring device and/or apparatus and/or at least one liquid measuring device and/or water content detection method and/or liquid content detection method and/or moisture detection method and/or a chemical and/or physical method for detecting the condition and/or at least one comparative pattern or sample and/or color pattern detection method sample and/or comparative sample etc., or the like, is used for the detection, to acquire data and/or information for this purpose and/or to fulfill one or more of the purposes according to the invention.

10. The method according to claim 1, for the purpose of a comparison between the metrical detection or metrical method and the visual detection or visual methods and/or for the transfer of the results of the one method into the other method and/or of the one detection into the other detection, comprising:
equipment-specific detection of the comparative patterns or, for example, of patterns of a common (tooth) color palette, materials samples same described, patterns according to one or more of the aforementioned claims and/or according to the purpose of determining the time of death and/or newly generated color patterns and/or comparative sample, etc.
use of the data detected in such a manner for the newly visually determined patterns or samples at the tooth and/or “object”, which is to be examined, e.g. to describe the visual detection of the patterns or samples in values, e.g. color measured values.

11. A method using the data detectable according to claim 1 during the water release chronology and/or the water absorption chronology and/or the liquid release chronology and/or the liquid absorption chronology for the identification of teeth and/or tooth material and/or living beings (e.g. humans, persons, animals, individuals, etc.) by means of the individual tooth-specific and/or substance-specific changes or developments comprising:
comparison of the newly-detected data at the tooth and/or object, which is to be detected and/or examined, with the already detected data with reference to the time factor or the comparison of the newly detected data or curve(s) or value development at the tooth and/or object, which is to be detected, along the liquid release and/or liquid absorption method with already detected data or curves or value development.

12. A color pattern palette and/or comparative samples comprising the expansion of the common (tooth) color pattern palettes into additional color regions, in regions or higher and/or lower brightness and/or stronger and/or less stronger saturation and/or by additional color hues with corresponding additional color patterns and/or developing of one or more of comparative samples, which correspond with or was related to the condition or the conditions of one or more of process-related point(s) of time and/or with a (one) condition or more conditions, depending on the purpose of the later use with and/or without assignment of the time factor.

13. The color pattern palette according to claim 12 with systematic arrangement and expansion of the color patterns, according to a (tooth) color space, comprising colors, which can be found in dry to liquid-saturated or liquid-carrying teeth and/or substances.

14. The color pattern palette according to claim 12 with systematic arrangement and expansion of the color patterns, arranged according to the (tooth) color spaces, which are assigned or can be assigned to points of time and/or to time differences and/or to liquid content conditions along the liquid release and/or the liquid absorption chronology and/or to the process of changing the condition.

15. The color pattern palette according to claim 12 with samples, embodied under inclusion of the “layering phenomenon”.

16. The color pattern palette according to one claim 12, wherein corresponding water contents and/or liquid contents and/or moisture level and/or conditions can be or are substance-specifically assigned to the color patterns and/or the color pattern group.

17. The color palette according to claim 12, wherein the color samples are assigned to the corresponding points of time and/or to the corresponding time and/or to the defined conditions and/or to the defined conditions within this process in systematic arrangement according to the condition and/or the chronological temporal material color space, substance color space or object color space.

18. The color palette according to claim 12, comprising a storage case for the aforementioned, e.g., shade guides, color patterns, color samples, comparative samples, etc. or the like, and wherein, with the storage case, potential components, e.g. time-specific color space palettes, color patterns can be stored in an orderly manner, e.g. according to time (sections), condition, liquid contents of the tooth or object, etc. and/or wherein the storage can be maintained in a locally stable manner by means of the storage case.

19. A detection system, in particular for the method according to claim 1 for detecting at least one sample, preferably several samples wherein the sample (e.g. tooth, teeth, material, substance, object, article, structure, etc.) and/or a region hereunto on this can be detected with a measuring process comprising a solid and/or reproducible relation between sample and at least one detection-qualified “instrument” (e.g. device, apparatus, equipment, instrument, detection system, sensor, detector, detection unit, detection element, elements qualified for detecting conditions, detection possibilities, detection element, etc., or the like) or the possibility to reproduce reproducibility of this relation, whereby sample(s) and/or regions on this can hereunto be detected simultaneously and/or successively, for example by means of a metrological detection.

20. A detection system, in particular according to claim 19, comprising a practical and/or sensible arrangement of the samples and/or of the regions and/or of the measuring surfaces (e.g. next to one another side by side, in series, on a surface, in a space, etc.) for the purpose of their presentation to the instrument.

21. The detection system according to claim 19, comprising a meaningful arrangement of the detection-capable instruments and/or parts thereof.

22. The detection system according to claim 19, comprising data acquisition via the (color) detection by means of sensors and/or detectors and/or color detection equipments (e.g. tristimulus device, spectral photometer, etc.) and/or camera and/or camera systems and/or by means of image recording (system) and/or laser detection.

23. The detection system according to claim 19, comprising the presence of a different instrument.

24. The detection system according to claim 19, according to which the detection takes place via optical fibers, for example via fiberglass cables and, for the purpose of detecting samples (e.g. color detection, spectral analysis, etc.), the one end thereof is in contact with the sample(s) and the other end is in contact with the instrument, for example.

25. The detection system according to claim 19, according to which, for example one instrument (e.g. light receiver, sensor, detector, a camera, camera system, image detection system, photocell and/or a component thereof, etc. or the like) is present on or above each sample and/or, however, on or above at least one sample and/or one or more samples are illuminated.

26. The detection system according to claim 19, according to which the samples are in fixation.

27. The detection system according to claim 1, according to which one or more sample(s) is/are stored in a liquid-tight and/or air-tight container and/or device element and/or the container and/or device element has an air access or liquid access and/or outlet and/or can be covered and/or sealed.

28. The detection system according to claim 19, according to which a sample or several samples isolated are subjected to simulated situations (e.g. milieu, climate, liquid quantity, liquid consistency, temperatures, etc.) and/or can be artificially aged.

29. The detection system according to claim 19, according to which the content of the container and/or of the device element can be influenced and/or controlled in the milieu and/or in the climate, and/or at least one determined (e.g. desired, used, simulated) milieu and/or climate can be established and/or maintained and/or can alter and/or change and/or can be controlled and/or this occurs according to a corresponding program and/or specifications and/or software, for example.

30. The detection system wherein several samples and/or regions can be simultaneously and/or successively determined, for example by means of a measuring process.

31. The detection system according to claim 30 according to which the sample-related data can be only isolated on the post-connected level downstream from the detection instrument and/or can be assigned to the sample.

32. The detection system according to claim 30, according to which the system comprises independent instruments (e.g. devices, equipments, systems, etc.), for example, and/or which can also be centrally networked, for example.

33. The detection system according to claim 30, according to which the simulation region and/or milieu and/or climate space allows for the storage of several samples and/or to subject them to other detection conditions and/or is accessible.

34. The detection system, in particular according to claim 30, according to which the detections for the data acquisition are supplemented by or exclusively comprise known water content methods and/or liquid content methods and/or moisture detection methods and/or condition detection methods and/or -instruments (e.g. thermo-gravimetrical analyses, gravimetrical analyses, NIR spectroscopy, conductivity measurements, etc.), in that, for example, each unit and/or several of joint units and/or each sample and/or group of samples is provided with at least one corresponding instrument.

35. The detection system, in particular according to claim 30, according to which, the data on the basis of the detections (e.g. color detection, the analysis of the spectral composition of the light and/or of other electromagnetic radiation reflected by the material and/or substance and/or other electromagnetic radiation, the course of radiation, etc.) are correspondingly assigned to the data from one or more of the known or common methods for liquid content measurements and/or moisture measurements and/or condition detection (e.g. infrared measurement, conductivity measurement, gravimetrical and/or thermogravimetrical analyses, chemical, physical analyses, etc.) and/or to process-related point(s) of time, so that reference data are now gaged or calibrated to substance-specific, process-related points of time and/or liquid contents and/or water contents and/or moisture and/or conditions.

36. The detection system, in particular according to claim 30, comprising the detection of data (e.g. color detection, color determination, the analysis of the spectral composition of the reflected, passed light, of the electromagnetic radiation, of the course of radiation, known methods for water content measurement, liquid content measurements, moisture measurements, condition detection, etc.) at an “object” (e.g. tooth, material, substance, object, etc.), which is to be examined or measured.

37. The detection system, in particular according to claim 30, according to which this system operates in non-contact mode and/or possesses a possibility for manual adjustment and/or for automatic self-adjustment of the sharpness and/or of the focus and/or a positioning automatic for the optimal measuring position of the object in relation to the instrument.

38. The detection system according to claim 30, according to which this system (for the detection) is used for the determination of the time of death and/or for the identification of data and/or of persons and/or of living beings and/or for gaging and/or calibration (conversion of data detected by means of a detection method and/or detection instrument into data detected by means of other detection methods and/or instruments) and/or for one or more of the aforementioned method purposes and/or the system is automated or is a machine.

39. An instrument, wherein the data, which can be acquired by this instrument at one or more of teeth and/or substances or the like are gaged or calibrated to corresponding process-related points of time and/or conditions and/or water and/or liquid contents and/or moisture of teeth and/or substances, and wherein the instrument detects at one tooth or more teeth and/or substance(s), which are to be examined or detected.

40. An instrument and/or detection system, wherein it is portable or permanently installed or non-portable.

41. A method and/or instrument and/or detection system, using a neuronal system for the detection or acquisition and/or processing of data, etc.

42. The method according to claim 1, wherein the data and/or data relationships obtained by means of the detection system or detection machine is brought into compliance with measuring data of any instrument, which can now fulfill one or more of the method purposes by itself.

43. The method according to claim 1, wherein the control of the operations and/or of the processes and/or of the simulation and/or of the adjustment of the conditions and/or the measurements and time intervals and/or evaluation occurs via a computer setting and/or software and/or via a setting by the manufacturer and/or by the user and/or via a neuronal network, etc.
